SEQUENCE LISTING (7 sequences)

SEQ ID NO: 1 - DNA, 20 bp, synthetic construct
  source 1..20: mol_type = other DNA
accacagtccatgccatcac

SEQ ID NO: 2 - DNA, 20 bp, synthetic construct
  source 1..20: mol_type = other DNA
aaaccagagattcccacacg

SEQ ID NO: 3 - DNA, 20 bp, synthetic construct
  source 1..20: mol_type = other DNA
ggatgaatctgacgaatctc

SEQ ID NO: 4 - DNA, 20 bp, synthetic construct
  source 1..20: mol_type = other DNA
tccaccaccctgttgctgta

SEQ ID NO: 5 - DNA, 20 bp, synthetic construct
  source 1..20: mol_type = other DNA
gtcgtgggtcttcctgtagc

SEQ ID NO: 6 - DNA, 20 bp, synthetic construct
  source 1..20: mol_type = other DNA
gcatcaggatactgttcatc

SEQ ID NO: 7 - protein, 7 aa, Synthetic construct
SVVYGLR

(19) United States
(12) Patent Application Publication
(10) Pub. No.: US 2023/0272058 A1
(43) Pub. Date: Aug. 31, 2023

(54) COMPOSITIONS AND METHODS FOR PREVENTING ACUTE KIDNEY INJURY-INDUCED ACUTE LUNG INJURY (AKI-ALI)

(71) Applicant: Washington University, St. Louis, MO (US)

(72) Inventor: Andreas Herrlich, St. Louis, MO (US)

(73) Assignee: Washington University, St. Louis, MO (US)

(21) Appl. No.: 17/858,065

(22) Filed: Jul. 5, 2022

Related U.S. Application Data
(60) Provisional application No. 63/218,183, filed on Jul. 2, 2021.

(51) Int. Cl. C07K 16/24

(52) U.S. Cl. CPC C07K 16/24

(57) ABSTRACT

A method of preventing or treating an acute kidney injury-induced acute lung injury (AKI-ALI) in a subject in need by administration of a therapeutically effective amount of an osteopontin inhibitor is disclosed.

### CROSS-REFERENCE TO RELATED APPLICATIONS

This application claims priority from U.S. Provisional Application Ser. No. 63/218,183 filed on Jul. 2, 2021, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer readable form comprising nucleotide and/or amino acid sequences of the present invention entitled ‘019663-US-NP_SEQ_LISTING_ST26.XML’, created on 4/12/2023, and sized at 7,298 bytes. The subject matter of the Sequence Listing is incorporated herein by reference in its entirety.

### FIELD OF THE DISCLOSURE

The present disclosure generally relates to compositions and methods for treating acute kidney injury-induced acute lung injury (AKI-ALI).

### BACKGROUND OF THE DISCLOSURE

Acute kidney injury has a mortality of 15-30%. Combined with acute lung injury mortality is 60-80%. Elevated OPN levels in ICU patients, many with acute kidney injury, are associated with an increased need for ventilation and increased mortality. No specific treatments are available to date.

Homeostasis in multicellular organisms and the response to its disturbance by injury is coordinated by cell-cell communication aimed at reestablishing homeostasis. Studies on cellular functions in health and disease to date mostly focus on understanding cell-cell communication and its mediators within a given organ or diseased tissue. However, mediators of interorgan crosstalk between cells in different organs or tissues that drive multiorgan failure in complex diseases with high mortality, their cellular sources and target cells are largely unknown. Clinically important examples of such interorgan crosstalk include acute-kidney-injury (AKI)-induced acute-lung-injury (AKI-to-ALI) with respiratory failure, COVID19-induced lung injury followed by AKI (ALI-to-AKI), or lung transplant-induced AKI (ALI-to-AKI), and suggest bi-directionality of the signaling process. AKI is a common problem and AKI develops in 2-5% of patients during hospitalization, in 50% of intensive care unit (ICU) patients, and about 20% of kidney transplant patients within the first 6 months after transplantation. Irrespective of its cause, AKI alone has a 15-30% mortality, which rises to 60-80% when AKI induces remote secondary organ complications (multiorgan failure), in particular AKI-to-ALI. Molecular mechanisms and mediators of AKI-to-ALI are not yet well understood. Candidate AKI-to-ALI mediators were largely derived from global gene knockout studies or the use of systemic interventions. Such studies implicated interleukin-6 (IL6) and tumor-necrosis-factor (TNF), as well as IL10 released from splenic CD4 T-cells as putative mediators. Mediators are thought to be released from the injured kidney, but this has not been conclusively shown, and kidney cell types that release and lung cell types or immune cells targeted by these mediators are unknown. Hormones, growth factors, chemokines, cytokines, neurotransmitters, and other secreted proteins (in the following referred to as ligands) act in cell-cell communication in autocrine/paracrine fashion locally or may have distant endocrine effects if they are released into the circulation. Ligand-induced cognate receptor activation in receiving cells then generally results in altered gene expression and phenotypic changes. To date, ligand-receptor (LR) pairing analysis using bulk or single-cell/single nucleus mRNAseq (scRNAseq or snRNAseq) gene expression datasets has been used to infer cell-cell communication within a local tissue or organ.

### SUMMARY OF THE DISCLOSURE

In various aspects, a method of treating or preventing an acute kidney injury-induced acute lung injury (AKI-ALI) in a subject in need by administration of a therapeutically effective amount of an osteopontin inhibitor is disclosed. In some aspects, the osteopontin inhibitor may be an anti-OPN-antibody. In some aspects, administration of the therapeutically effective amount of the osteopontin inhibitor results in inhibition of lung vascular leakage and inflammation.

In various other aspects, a method of treating or preventing a multi-organ failure associated with an acute tissue injury in a subject in need by administration of a therapeutically effective amount of an osteopontin inhibitor is disclosed. In some aspects, the osteopontin inhibitor comprises anti-OPN-antibody.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way. The patent or application file contains at least one drawing executed in color. Copies of this patent or patent application publication with color drawing(s) will be provided by the Office upon request and payment of the necessary fee.

FIG. 1A is a schematic of the AKI-ALI experimental scheme.

FIG. 1B is a graph of serum BUN at Sham, Day 1, Day 3, Day 5 after AKI.

FIG. 1C is a graph of serum creatine values (bottom) at Sham, Day 1, Day 3, Day 5 after AKI.

FIG. 1D are images of H&E stained lung of Sham and injured lung Day 1, Day 3, and Day 5 after AKI.

FIG. 1E is a graph of alveolar thickness measurements of Sham, and injured lung Day 1, Day 3, and Day 5 after AKI.

FIG. 1F are images of lung neutrophils (Ly6G+), alveolar (CD68high) and interstitial macrophages (CD68low), along with a graph of the quantification of cell type density in Sham lungs, as well as lungs Day 1, Day 3, and Day 5 after AKI.

FIG. 1G is a set of graphs of % CD45+ cells in lung neutrophils (CD45+, Ly6G+), alveolar (CD45+, CD68high, Siglec-F−) and interstitial macrophages (CD45+, CD68low, Siglec-F−) Day 1 after Sham or AKI using CyTOF.

FIG. 1H is a set of graphs of serum BUN elevations and BAL fluid protein/albumin after sham or AKI, day 1. Total protein was detected by the Pierce BCA Protein Assay. Albumin was detected by enzyme-linked immunosorbent assay (ELISA).

FIG. 1I is a set of graphs showing total immune cells, macrophages, and neutrophils from Sham and injured lung as detected by fluorescence-activated cell sorting (FACS).

FIG. 1J is a set of graphs of pO2 from an arterial blood gas in sham and treated animals 1 day after treatment.

FIG. 1K are images of H&E stained lungs day 1 after Sham or nephrectomy (Nx).

FIG. 1L is a graph of alveolar thickness Day 1 after Sham or nephrectomy (Nx).

FIG. 1M is a set of graphs of % CD45+ cells in lung neutrophils (CD45+, Ly6G+), alveolar (CD45+, CD68high, Siglec-F−) and interstitial macrophages (CD45+, CD68low, Siglec-F−) Day 1 after Sham or nephrectomy (Nx).

FIG. 2A is a schematic of the scRNAseq experimental scheme of kidney and lung in setting of AKI-induced ALI (AKI-ALI).

FIG. 2B is a graph of serum BUN values Day 1 after Sham or AKI.

FIG. 2C is a graph of the Seurat object of combined kidneys from sham and AKI treated animals.

FIG. 2D is a dotplot of marker genes in the kidneys broken down by cell types.

FIG. 2E is a graph of the Seurat object of combined lungs from sham and AKI treated animals.

FIG. 2F is a dotplot of marker genes in the lungs broken down by cell types

FIG. 2G is a graph of the Seurat object from kidneys of Sham and AKI treated animals and a graph summarizing a quantification of neutrophils, monocytes, and macrophages in sham or AKI samples expressed as the percentage of total cells detected by Seurat.

FIG. 2H is a graph of the Seurat object from lungs of Sham and AKI treated animals and a graph summarizing a quantification of neutrophils, monocytes, and macrophages in sham or AKI samples expressed as the percentage of total cells detected by Seurat.

FIG. 3A is a schematic of the experimental design of the ligand-receptor (L-R) linkage analysis across organs.

FIG. 3B is an L-R linkage analysis heatmap between kidney stromal (non-immune) cells and lung immune cells 1 day after treatment from Sham (panel 1) and AKI (panel 2) treated animals.

FIG. 3C is an L-R linkage analysis heatmap between kidney stromal (non-immune) cells and lung stromal cells 12 days after treatment from Sham (panel 1) and AKI (panel 2) treated animals.

FIG. 3D is an L-R linkage analysis heatmap between kidney immune cells and lung immune cells 1 day after treatment from Sham (panel 1) and AKI (panel 2) treated animals.

FIG. 3E is an L-R linkage analysis heatmap between kidney immune cells and lung stromal cells 1 day after treatment from Sham (panel 1) and AKI (panel 2) treated animals.

FIG. 4 is a gene ontology (GO) term analysis of ligands included in the top 30% L-R pairings for sham and injured animals.

FIG. 5A is heatmap summarizing a L-R pairing analysis of kidney stromal (non-immune) and immune cells 4 hours after AKI to lung immune cells Day 1 after AKI (upper panel) or kidney stromal (non-immune) and immune cells 4 hours after AKI to lung non-immune cells Day 1 after AKI (lower panel).

FIG. 5B is heatmap summarizing a L-R pairing analysis of kidney stromal (non-immune) and immune cells 12 hours after AKI to lung immune cells Day 1 after AKI (upper panel) or kidney stromal (non-immune) and immune cells 4 hours after AKI to lung non-immune cells Day 1 after AKI (lower panel).

FIG. 6A is a set of 2 violin plots of osteopontin (OPN) scRNAseq expression data from the kidney 1 Day after Sham or AKI treatment.

FIG. 6B is a dot plot of OPN snRNAseq expression data in kidney for sham, 4 hr, 12 hr, Day 2, Day 14, and Week 16 conditions.

FIG. 6C is a set of 2 violin plots of osteopontin (OPN) scRNAseq expression data from the lung 1 Day after Sham or AKI treatment.

FIG. 6D is a schematic of the experimental design of the OPN scRNAseq experiments.

FIG. 6E is a graph of serum BUN from Sham treated animals and animals 1 hr, 2 hr, 4 hr, 6 hr, 12 hr, 1 day, 3 days, and 5 days after AKI treatment.

FIG. 6F is a graph of serum creatine from Sham treated animals and animals 1 hr, 2 hr, 4 hr, 6 hr, 12 hr, 1 day, 3 days, and 5 days after AKI treatment.

FIG. 6G is a graph of serum KIM-1 from Sham treated animals and animals 1 hr, 2 hr, 4 hr, 6 hr, 12 hr, 1 day, 3 days, and 5 days after AKI treatment.

FIG. 6H is a set of 2 graphs of qPCR expression data of OPN in kidneys (left) and lungs (right) 1 hr, 2 hr, 4 hr, 6 hr, 12 hr, 1 day, 3 days, and 5 days after AKI as compared to Sham.

FIG. 6I is a graph of OPN concentration in serum from ELISA from Sham treated animals (D0) and 1 hr, 2 hr, 4 hr, 6 hr, 12 hr, 1 day, 3 days, and 5 days after AKI.

FIG. 6J is a set of 3 optical images showing lung deposition of injected fluorescently marked OPN after Sham treatment (1 hour) or 30 min-1 hour after AKI treatment.

FIG. 6K is a set of optical images showing colocalization of OPN Alexa Fluor 555 (Alexa 555) with CD68+/CD44+ alveolar macrophages (fat arrows) and IMs (thin arrows) but not with Ly6G+ neutrophils. Arrows show colocalization with CD68/CD44.

FIG. 7A is a dot plot of Scc1 expression over time in the kidney after AKI.

FIG. 7B is a dot plot of Scc1 expression in the lung of different cell types in Sham and AKI treated animal.

FIG. 7C is a graph of the concentration of OPN in kidneys over time after AKI.

FIG. 7D contains a set of optical images from kidneys stained for CD31 and OPN in kidneys of Sham treated animals and animals treated with AKI (30 minutes and 1 hour post treatment).

FIG. 7E is a graph of expression levels of various factors over time (bottom).

FIG. 8A is a set of H&E stained images of lungs treated with AKI over time.

FIG. 8B is a graph of alveolar wall thickness from lungs of mice treated with AKI at different time points post-treatment.

FIG. 8C is a set of optical images of lung immune cells stained with Ly6G and CD68 taken from mice treated with AKI at different time points post treatment (left), along with corresponding graphs quantifying the amount of each marker at each time point (right).

FIG. 8D is a set of images of lungs stained with Evans blue from mice at different time points of post-AKI treatment.

FIG. 8E is a graph of the amount of Evans blue found in the lungs described in FIG. 8D.

FIG. 8F contains lung electron microscopy images and quantification of endothelial tight junction length (in nanometers) at day 1 in sham or day 1 after AKI in wt control or OPN-global KO mice.

FIG. 8G is a dot plot from scRNAseq of lung endothelial cells 1 day after sham (LS) or AKI (rLI) treatment, along with a graph of expression levels of ZO-1 from the same lungs based on immunohistochemical staining.

FIG. 8H is a set of optical images of lung ZO-1 protein expression [immunofluorescence (IF)]: ZO-1 (red), CD31 (green, marking endothelial cells), DAPI (blue, marking nucleus), and quantification day 1 after sham or AKI.

FIG. 8I is a set of optical images of lung endothelial cells stained for ZO-1, DAPI, and CD31 1 day after sham or AKI treatment.

FIG. 9A is a schematic of the experimental setup of the global OPN knockout or anti-OPN antibody neutralization experiments.

FIG. 9B is a graph of serum BUN levels in the models 1 day after AKI treatment.

FIG. 9C is a graph of serum KIM-1 levels in the models 1 day after AKI treatment.

FIG. 9D is a set of images of H&E stained lungs from mice 1 day after AKI treated with IgG antibody or neutralizing antibody, along with w.t. and knockout models.

FIG. 9E is a graph of the alveolar wall thickness quantified from the H&E images described in FIG. 9D.

FIG. 9F is a set of immunofluorescence images of lung immune cells from the models described in FIG. 9A, along with a graph of expression of Ly6G in the various models.

FIG. 9G is a set of 2 images of lungs stained with Evans blue from mice with AKI treated with either IgG antibody or OPN neutralizing antibody.

FIG. 9H is a graph quantifying the amount of Evans blue found in the lungs described in FIG. 9G.

FIG. 9I is a graph quantifying the arterial blood oxygen partial pressure after Sham or AKI, day 1.

FIG. 10A is graph of OPN serum levels in AKI patients.

FIG. 10B is a graph correlating OPN concentration and creatinine concentration in patients.

FIG. 10C is a graph correlating OPN concentration and BUN concentration in patients.

FIG. 11 contains a bar graph of expression of various factors from the lungs of mice 1 day after AKI treated with either IgG (white bars) or OPN neutralizing antibody (grey bars), as well as from the lungs of OPN KO mice (black bars), as well as dot plots summarizing scRNAseq-measured expression of various factors lung endothelial cells 1 day after sham (LS) or AKI (rLI) treatment.

FIG. 12A is an experimental scheme wherein w.t. mice with and without injury are injected with OPN.

FIG. 12B is a graph of a graph of serum ELISA-measured OPN protein levels 6 hours after sham, severe AKI (positive control), or mild AKI.

FIG. 12C is a graph of a graph of serum ELISA-measured BUN 6 hours after sham, mild AKI±OPN injection, or severe AKI (positive control).

FIG. 12D is a set of images of lung hematoxylin and eosin stained 6 hours after sham, mild AKI±OPN injection, or severe AKI (positive control).

FIG. 12E is a graph of the alveolar wall thickness quantified from the H&E images in FIG. 12C.

FIG. 12F is a set of immunofluorescent images of lung immune cells from mice receiving an i.p. injection vehicle or OPN, along with correlating graphs quantifying the amount of Ly6G and CD68 found in the immune cells from the 2 groups of mice.

FIG. 13A is a schematic of the experimental setup for the transplantation of ischemic OPN KO kidneys.

FIG. 13B is a graph of kidney qPCR-measured KIM-1 expression in WT-WT and OPN KO-WT day 1 after transplant.

FIG. 13C is a graph of the tubular damage score in lungs of WT-WT and OPN OK-WT knockout mice in the experiment described in FIG. 13A.

FIG. 13D is a graph of serum OPN concentration by ELISA in transplant experiments.

FIG. 13E is a set of images of lungs stained with Evans blue from mice in the transplant experiments.

FIG. 13F is a graph of Evans blue content in the lungs described in FIG. 13E.

FIG. 13G is a set of images of H&E stained lungs from mice in the transplant experiments described in FIG. 13A.

FIG. 13H is a graph of the alveolar wall thickness quantified from the H&E images from FIG. 13G.

FIG. 13I is a set of immunofluorescent images of immune cells from transplant mice and corresponding graphs quantifying the amount of Ly6G+ and CD68+ cells in the images.

FIG. 13J is a graph of expression of CD68 in immune cells from mouse lungs for the experiments described in FIG. 13A.

FIG. 14 is a graph of the Seurat object from lungs of Sham and AKI treated animals.

FIG. 15 is a plot of the 50 most differentially expressed genes between sham and AKI treated mice.

FIG. 16A is a graph comparing serum BUN levels in AKI mice treated with either vehicle or a CCR2 inhibitor.

FIG. 16B contains a set of immunofluorescence images of AKI mice treated with either vehicle or a CCR2 inhibitor.

FIG. 16C is a graph quantifying macrophage content based on the immunofluorescence images of FIG. 16B.

FIG. 17A is a schematic diagram illustrating an experimental scheme of ischemic kidney transplantation, in which wt kidneys were transplanted into wt or OPN-global KO mice (WT-WT or “WT-OPN KO”).

FIG. 17B is a series of images of hematoxylin and eosin stained lungs at day 1 after transplant for WT-WT and WT-OPN KO. Sham lung and OPN KO-WT are shown for comparison.

FIG. 17C is a graph summarizing alveolar wall thickness measurements on day 1 after transplant.

FIG. 17D is a series of immunofluorescence images showing lung neutrophils (Ly6G+, green), alveolar macrophages (CD68high, large, red), and IMs (CD68low, small, red) and quantification in lungs of sham-operated mice, WT-WT, OPN KO-WT, or WT-OPN KO day 1 after transplant. DAPI stain (blue) was used to visualize nuclei.

FIG. 17E is a series of immunofluorescence images showing colocalization of endogenous OPN released from transplant with CD68+/CD44+ alveolar macrophages (fat arrows) and IMs (thin arrows) but not with Ly6G marker (neutrophils). Arrows show colocalization with CD68/CD44. n=4 to 7 animals per measurement. *P<0.05, **P<0.01, ***P<0.001, and ###P<0.005.

FIG. 18A is a schematic illustration of an experimental scheme that includes vehicle control of OPN protein i.p. injection into mice without AKI.

FIG. 18B is a graph summarizing serum BUN in vehicle or OPN injected mice at Day 1.

FIG. 18C contains images of H&E stained lungs in vehicle or OPN injected mice at Day 1.

FIG. 18D is a graph summarizing alveolar wall thickness measurements in vehicle or OPN injected mice at Day 1.

FIG. 18E contains immunofluorescence images of lung neutrophils (Ly6G+, green), alveolar (CD68high, large, red) and interstitial macrophages (CD68low, small, red) and quantification in lungs of vehicle or OPN injected mice at Day 1. DAPI stain (blue) was used to visualize nuclei. n=3-5 animals per measurement, NS=non-significant

FIG. 19 contains immunofluorescent images of lung tissue after fluorescent OPN injection (OPN Alexa 555 (red) in lung tissue at 1 hour after Sham or 30 min-1 hours after AKI (injection at reperfusion or similar time in sham). The extracellular matrix component Nidogen (green) was used to localize extracellular/interstitial space. DAPI stain (blue) was used to visualize nuclei. Open arrows=OPN Alexa 555 accumulation in lung interstitium, closed arrows=OPN Alexa 555 co-localization with lung cells.

### DETAILED DESCRIPTION OF THE DISCLOSURE

The present disclosure is based, at least in part, on the discovery of a mediator of acute kidney injury-induced acute lung injury (AKI-ALI): kidney-released osteopontin (OPN or Spp1). As illustrated in the Examples below, blocking OPN with a neutralizing antibody reduces AKI-ALI lung injury. Mice with mild AKI show no lung injury and OPN injection reverses this protection and causes lung injury.

The examples herein describe the first ligand-receptor (LR) pairing analysis across different organs to successfully determine a mediator of interorgan crosstalk in multiorgan failure in vivo. The results of the examples identify kidney tubule cell-released circulating osteopontin (OPN or Spp1) as a mediator of AKI-induced remote acute-lung-injury with respiratory failure (AKI-ALI).

To identify interorgan cross-talk mediators, acute kidney injury (AKI)-induced acute lung injury (ALI) was used as a clinically important example as described herein. Using kidney and lung single-cell RNA sequencing after AKI in mice followed by ligand-receptor pairing analysis across organs, kidney ligands to lung receptors, kidney-released circulating osteopontin (OPN) was identified as a novel AKI-ALI mediator. OPN release from kidney tubule cells triggered lung endothelial leakage, inflammation, and respiratory failure. Pharmacological or genetic OPN inhibition prevented AKI-ALI. Transplantation of ischemic wt kidneys caused AKI-ALI, but not of ischemic OPN-global knock-out kidneys, identifying kidney-released OPN as necessary interorgan signal to cause AKI-ALI. It is shown that OPN serum levels are elevated in patients with AKI and correlate with kidney injury. The results demonstrate feasibility of using ligand-receptor analysis across organs to identify interorgan cross-talk mediators and may have important therapeutic implications in human AKI-ALI and multiorgan failure.

In various aspects, the disclosure provides a method of treating or preventing an acute kidney injury-induced acute lung injury (AKI-ALI) in a patient in need that comprises administering a therapeutically effective amount of an osteopontin inhibitor. Without being limited to any particular theory, circulating osteopontin is thought to cause lung endothelial leakage and to deposit itself into the lung interstitial space, resulting in chemotaxis of neutrophils and CCR2+ monocytes and chemoattractant expression in the lung. The osteopontin inhibitor may be any suitable compound including, but not limited to, an anti-OPN-antibody. Without being limited to any particular theory, the administration of the osteopontin inhibitor inhibits the mediation of lung vascular leakage and inflammation by osteopontin released by distal and proximal tubule cells of an acutely injured kidney.

The work using kidney transplantation provides the first conclusive evidence in vivo that a kidney-released circulating mediator is causally involved in secondary organ failure after kidney injury. Neutralization of circulating OPN or OPN-global KO prevented AKI-ALI and respiratory failure, and ischemic kidneys from OPN-global KO mice transplanted into wt mice failed to raise serum OPN levels and to induce AKI-ALI, as compared to transplantation of ischemic wt kidneys into wt mice. In turn, transplanted wt kidneys reversed the protection of OPN-global KO mice against AKI-ALI, revealing the critical necessity of kidney released OPN for the development of AKI-ALI. It is shown that distal tubule—and proximal tubule—released OPN is poised to act as a key mediator of lung vascular leakage and inflammation early in the lung's response to kidney injury. The fast response is due to OPN's baseline expression and quick up-regulation, causing lung vascular leakage and immune cell accumulation within an hour, ultimately resulting in functional impairment with reduced arterial oxygenation. This finding has implications beyond AKI, as tissue injury due to various causes can raise OPN serum levels in humans, including but not limited to direct lung injury by COVID-19, bacterial pneumonia, sepsis from various causes, or cardiac injury. OPN has also been described as a marker of kidney injury and declining kidney function, as example, but not much is known about the mechanisms involved. All these conditions are highly associated with multiorgan failure, particularly AKI and ALI or its most severe form, ARDS. This suggests that OPN up-regulation represents a conserved response to tissue injury in different organs and might link circulating OPN to secondary organ failure in general, as it is suggested by various studies of serum OPN as a biomarker of multiorgan failure mortality in humans. Clinically, AKI-ALI is likely underrecognized because of many confounders, such as coexisting hypervolemia or cardiac dysfunction, which often serve as primary explanations for respiratory distress in patients with AKI, with AKI-induced proinflammatory contributions underestimated. In this regard, our study provides important insights. However, it is very much clinically recognized that ARDS is negatively affected by the presence of AKI. In this context, alveolar macrophages in ARDS patients highly express OPN, and lung macrophages also express OPN in an experimental mouse model of ALI/ARDS. In addition, increased OPN levels in sputum or BAL have also been linked to inflammation and severity of disease in other human lung diseases, such as cystic fibrosis, chronic obstructive pulmonary disease, and asthma. Thus, OPN released by the AKI kidney may represent an important modifier of preexisting lung disease, particularly ALI/ARDS.

A study in mice connected OPN to lung injury after intestinal ischemia. However, in contrast to this disclosure, OPN was found up-regulated at comparably low levels in both the ischemic gut and the remotely injured lung, not allowing to identify the source of comparably low serum OPN elevations. OPN neutralization significantly improved gut injury but only mildly improved lung injury, suggesting that OPN does not represent a major causal link in gut injury-induced lung injury. A study using renal transplantation to induce ischemic AKI in rats showed that OPN is up-regulated in the lung and at rather low levels in serum at day 1 after transplantation; kidney was not assessed as an OPN source. OPN inhibition with hydrodynamic siRNA injection improved lung necroptosis and inflammation, but its effects on serum or lung OPN expression were not tested. Thus, similar to the study with intestinal ischemia, this study also did not identify OPN as a causal interorgan signal in AKI-ALI. Broadening the putative relevance of the study is the fact that remote organ effects ventilation, but proof of this would require new clinical trials designed to detect effects of serum OPN neutralization on respiratory outcomes. A study of critically ill patients with multiorgan failure including AKI requiring dialysis found that OPN levels in this cohort were significantly elevated when compared with critically ill controls without AKI, possibly indicating that the degree of kidney injury correlates with the degree of serum OPN elevation. The results show that serum OPN is elevated in cases of human AKI (no multiorgan failure or sepsis) and correlates with the degree of kidney injury, strengthening the link between elevated OPN serum levels and kidney injury in humans. Together, published data and the described results suggest that circulating OPN might have similar roles in AKI-ALI with respiratory failure in humans as detected in mice.

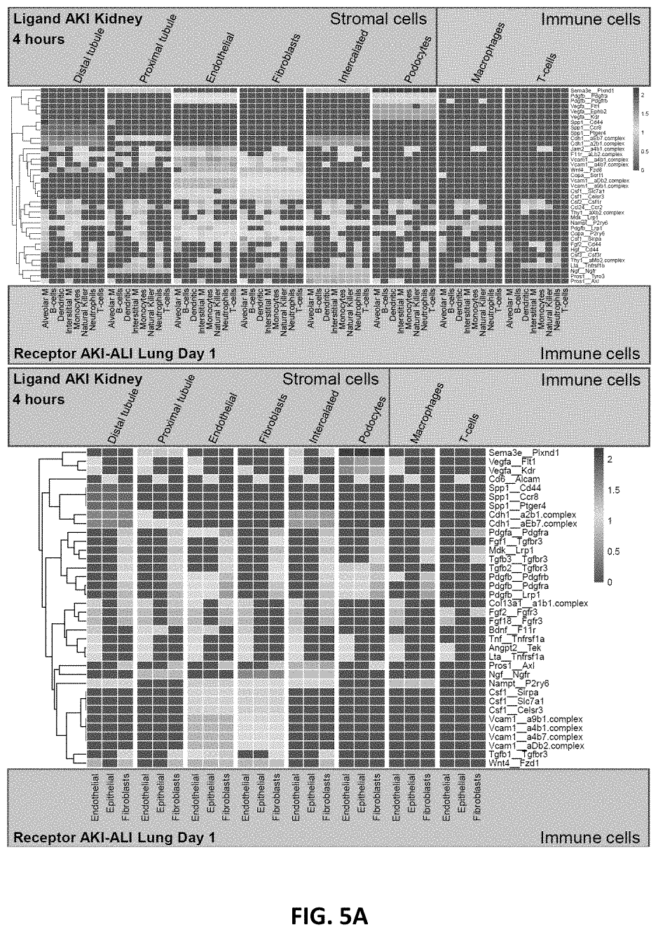

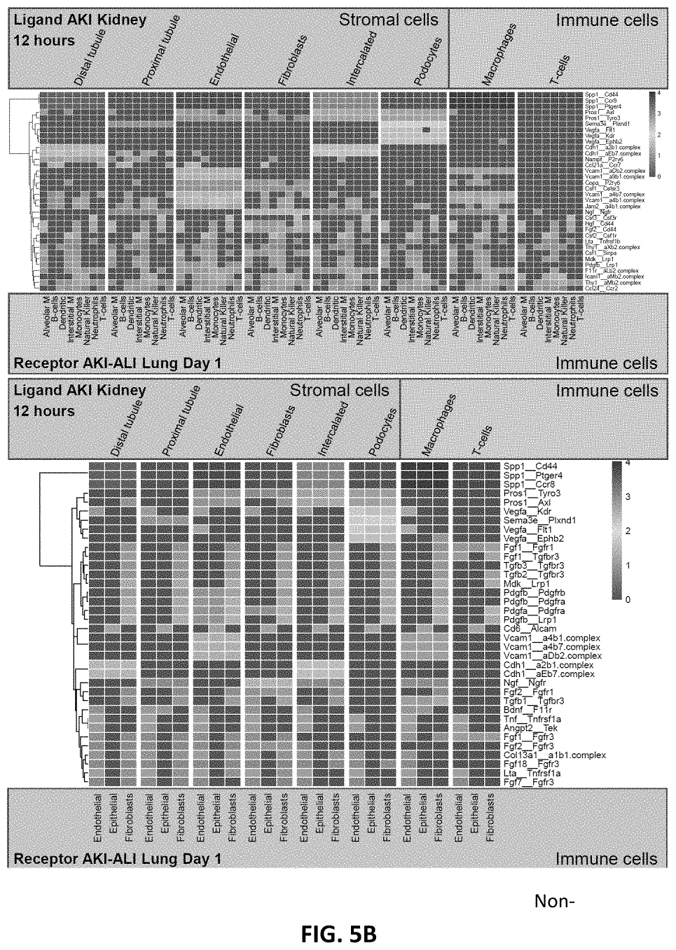

The studies also identify circulating OPN as a key regulator of endothelial barrier permeability in vivo in AKI-ALI. Disruption of the lung endothelial barrier and/or the pulmonary epithelial barrier is a common feature of ALI/ARDS. A number of other studies have also reported lung vascular leakage after AKI and lung endothelial cell activation. For the AKI-ALI mediator TNF, this may be related to TNFR1-dependent lung endothelial cell apoptosis. However, while IL6 and TNF inhibition have been reported to strongly reduce lung neutrophil accumulation after AKI, they only moderately reduce lung vascular leakage. How IL6 and TNF relate to OPN's ability to disrupt the endothelial barrier is unknown. A previous study associated OPN with endothelial barrier permeability in pulmonary vein endothelial cells (PUVECs) in primary culture. In this report, OPN expression was increased, and ZO-1 and Cldn5 expression levels were decreased in PUVECs in septic rats or in lipopolysaccharide-treated PUVECs isolated from these rats. Up-regulation of connexin43 in PUVECs stimulated OPN expression and down-regulated ZO-1 and Cldn5, increasing vascular permeability. OPN knockdown blocked this effect. These data are consistent with our findings of ZO-1 and Cldn5 down-regulation in single-cell expression data of lung endothelial cells (FIG. 5G) and down-regulation of ZO-1 protein in immuno-fluorescence stains of AKI-ALI lung tissue (FIG. 5H). In addition, principally consistent with our findings, OPN was found up-regulated by the host and tumor in a mouse model of malignant pleural effusions where OPN neutralization reduced leakage of fluid into the pleural space.

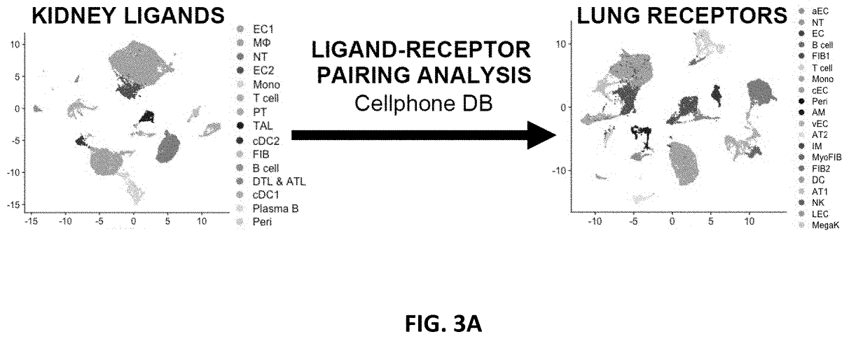

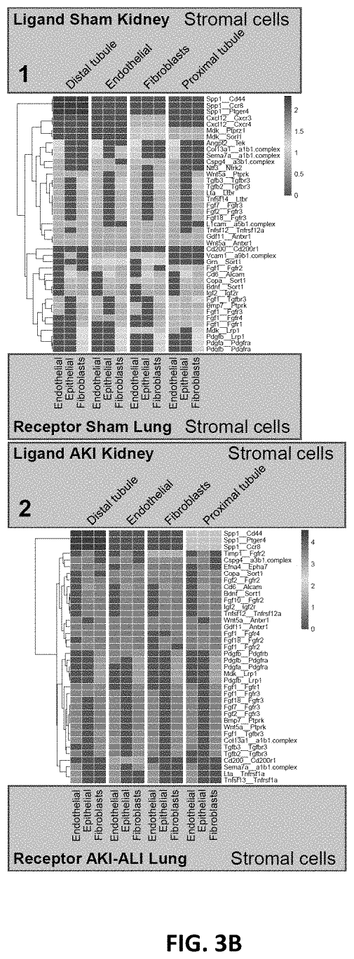

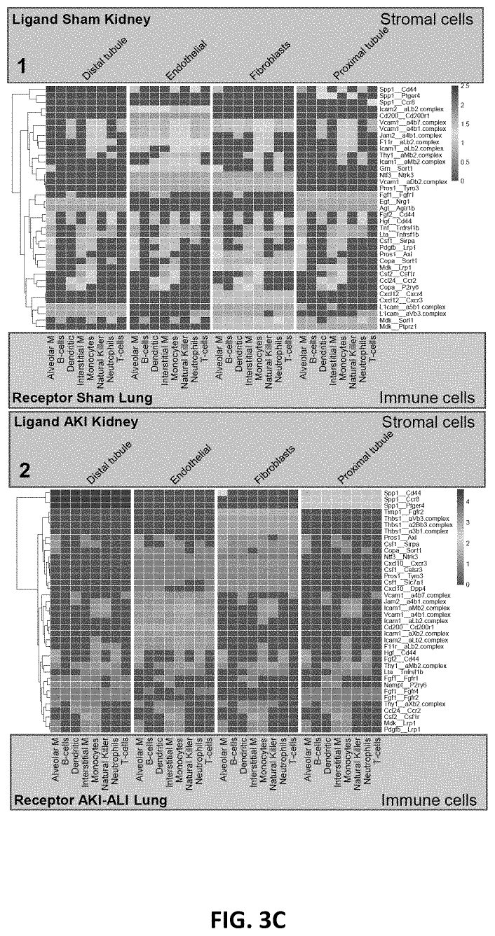

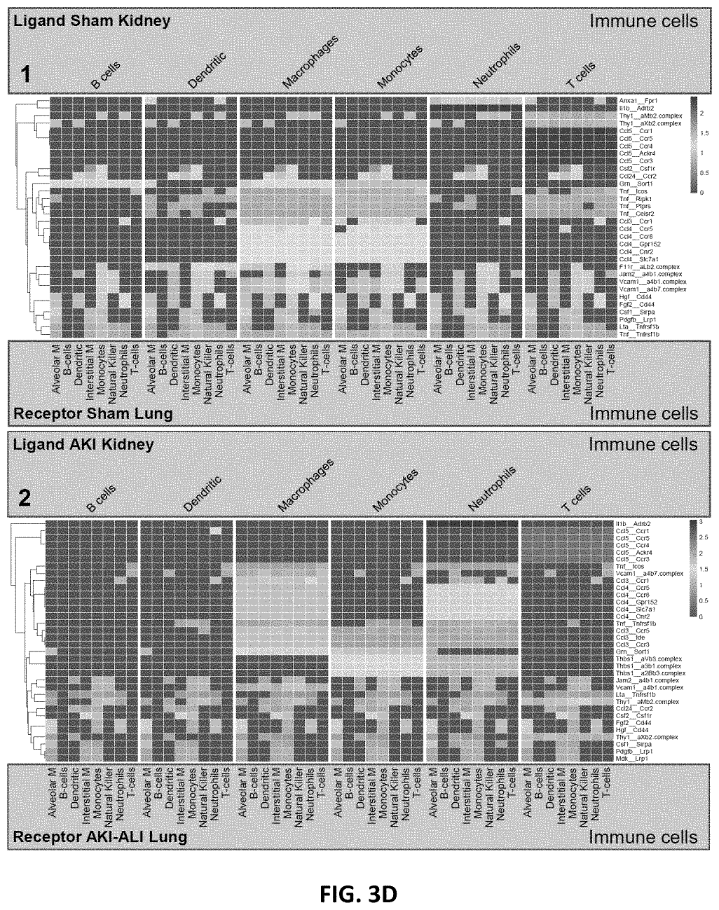

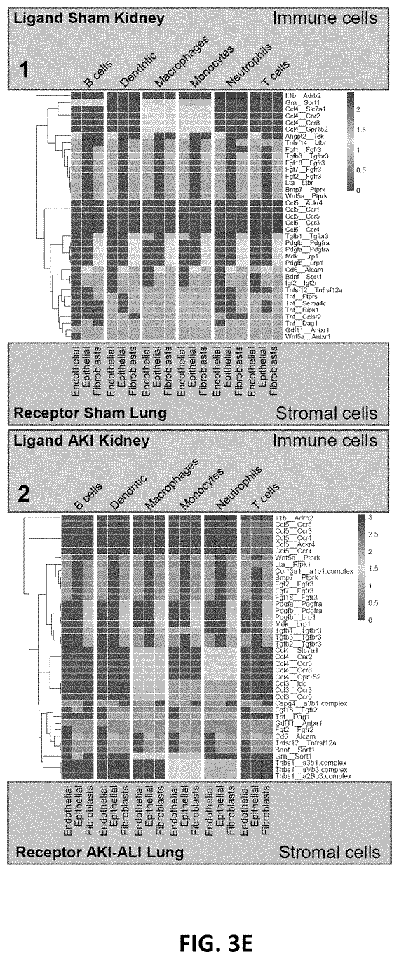

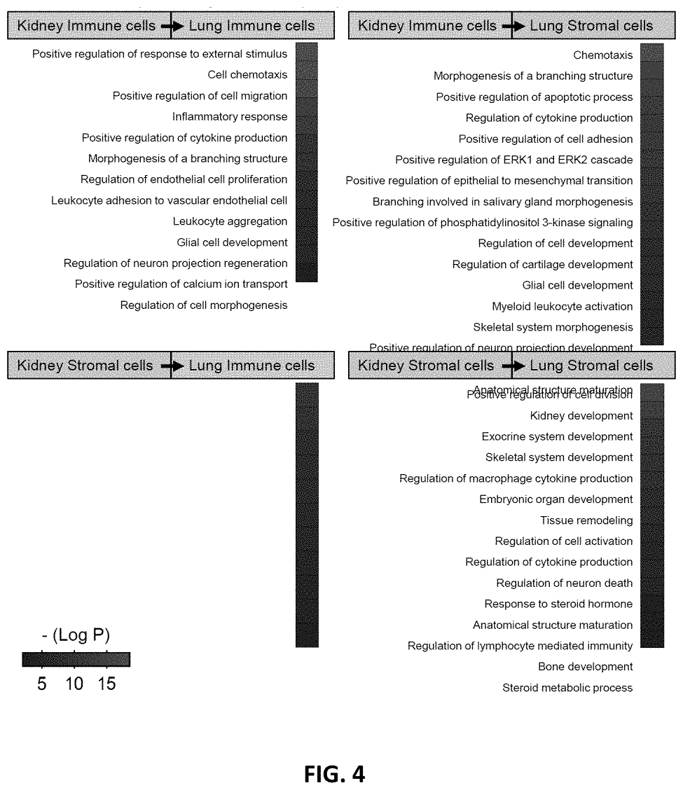

TNF and its family member lymphotoxin A were also found in our L-R pairing analysis, adding additional validity to our strategy and findings. Circulating TNF is thought to participate in the induction of lung endothelial cell death via its receptor TNFR1, as neutralization of TNF with etanercept in AKI-injured wt mice or use of global TNFR1 KO mice reduced lung cell apoptosis after AKI. Various moderate to low significance pairings of TNF with its cognate receptors was identified, particularly TNF:TNFR1, and other known interacting proteins. These TNF pairings were primarily identified between kidney immune cells and lung stromal or immune cells when pairing sham kidney with sham lung or at some-what higher significance when pairing early AKI kidney (4 and 12 hours) with day 1 AKI-ALI lung. However, most of these pairings lost significance when day 1 AKI kidney was paired with day 1 AKI-ALI lung (FIGS. 3D and E, panels 1 and 2, and fig. S2). This suggests that TNF plays a role early in the development of AKI-ALI, consistent with the role of TNF as a type 1 inflammatory cytokine known to act early in establishing inflammation, and this is also consistent with the referenced study of the effect of TNF inhibition/TNFR1 deficiency on AKI-ALI. Moderate to low significance L-R pairing of kidney TNF with its lung receptor TNFR2 (Tnfrsf1b) were also identified in immune cells but not stromal cells; however, the significance of these pairings did not increase with injury (FIG. 3B to D). Similar to TNF, IL6 was linked to AKI-ALI using IL6 neutralization and IL6 global KO mice. IL6 and its receptor, however, were not identified by the analysis.

Significant lung changes after bilateral nephrectomy were not found. Others have, however, demonstrated changes in gene expression profiles and lung inflammation after nephrectomy, and the elimination of AKI-induced cytokines can be delayed by nephrectomy. In the experiments, small changes in serum OPN can be detected even after sham surgeries, without kidney injury or nephrectomy, possibly induced by the relatively small tissue injury induced by sham surgery. It is thus possible that the detection of lung changes after nephrectomy depends, at least, in part, on the extent of tissue injury caused by the surgeon. This might explain some of the discrepancies between the described work and the work of others.

OPN, also known as SPP1, early T lymphocyte activation-1, or uroprotein is a member of the small integrin-binding ligand N-linked glycoprotein family proteins. OPN has, at least, two types of receptors, both broadly expressed in many cell types but highly up-regulated particularly in immune cells. It can interact with integrins via N-terminal domains and CD44 receptors via C-terminal domains. OPN interaction with CD44 causes chemotaxis of neutrophils and macrophages, whereas interactions with integrins relay immune cell spreading and activation signals. OPN (SPP1):integrin pairings were, however, not detected in the analysis. In the case of the two other predicted significant OPN (SPP1) L-R pairings, SPP1:CCR8 and SPP1:PTGER4 (prostaglandin E receptor 4)(FIG. 3B), it is unclear whether these interactions produce cellular signals, as these pairings were predicted by CellPhoneDB on the basis of protein-protein interaction data curated from various experimental approaches, but it is not known whether these interactions produce a cellular signal. Which target cells are affected by OPN in the model remains to be determined. In the lung scRNAseq dataset, CD44 is broadly expressed at low levels in lung stroma but robustly expressed in immune cells and highly up-regulated by injury in lung neutrophils, monocytes/macrophages, and T cells. This suggests that OPN might preferentially target immune cells rather than stromal cells of the lung.

In various aspects, circulating OPN released by the injured kidney was identified as a causal key mediator of lung endothelial leakage, lung edema, and lung inflammation with respiratory compromise. The human sample data and available published evidence suggest that therapeutic targeting of OPN or of its associated regulatory or target components should be evaluated in patients with multiorgan failure that are at high risk of ALI or that already have ALI, particularly in the presence of AKI. Any benefit in this area could lead to a meaningful if not substantial reduction of the very high mortality of multiorgan failure.

Osteopontin Modulation Agents

As described herein, osteopontin has been implicated in various diseases, disorders, and conditions. As such, modulation of osteopontin expression can be used for the treatment of such conditions. An osteopontin modulation agent can modulate osteopontin response or induce or inhibit osteopontin production. Osteopontin modulation can comprise modulating the expression of osteopontin by cells, modulating the quantity of cells that express osteopontin or modulating the quality of the osteopontin-expressing cells.

Osteopontin modulation agents can be any composition or method that can modulate osteopontin expression by cells (e.g., distal and proximal tubule cells of a kidney). For example, an osteopontin modulation agent can be an activator, an inhibitor, an agonist, or an antagonist. As another example, osteopontin modulation can be the result of gene editing.

An osteopontin modulation agent can be an anti-osteopontin antibody (e.g., a monoclonal antibody to osteopontin).

Osteopontin Signal Reduction, Elimination, or Inhibition by Small Molecule Inhibitors, shRNA, siRNA, or ASOs

As described herein, an osteopontin modulation agent can be used for use in anti-inflammatory therapy and/or to prevent or inhibit an acute kidney injury-induced acute lung injury (AKI-ALI). An osteopontin modulation agent can be used to reduce/eliminate or enhance/increase signals at osteopontin receptors of lung cells. Non-limiting examples of suitable osteopontin receptors include CCR8 and PTGER4. For example, an osteopontin modulation agent can be a small molecule inhibitor of CCR8 or PTGER4 signaling. As another example, an osteopontin modulation agent can be a short hairpin RNA (shRNA). As another example, an osteopontin modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

Osteopontin Inhibiting Agent

One aspect of the present disclosure provides for targeting of osteopontin, its receptor, or its downstream signaling. The present disclosure provides methods of treating or acute kidney injury-induced acute lung injury (AKI-ALI), based on the discovery that kidney-released osteopontin acts as a mediator of acute kidney injury-induced acute lung injury (AKI-ALI). The present disclosure further provides for inhibition of osteopontin released in association with a variety of other injuries or disorders including, but not limited to, acute kidney injury, direct lung injury by COVID-19 or pneumonia, acute respiratory distress syndrome (ARDS), sepsis, cardiac injury, and any other injury or condition associated with multiorgan failure without limitation.

As described herein, inhibitors of osteopontin (e.g., antibodies, fusion proteins, small molecules) can reduce or prevent lung failure associated with acute kidney injury or any of the other injuries or disorders described above associated with osteopontin-mediated multiorgan failure. An osteopontin inhibitor can be any agent that can inhibit osteopontin, downregulate osteopontin, or knockdown osteopontin.

As an example, an osteopontin inhibitor can inhibit CCR8 or PTGER4 signaling mediated by osteopontin released by distal and proximal tubule cells of an acutely injured kidney.

For example, the osteopontin inhibitor can be an anti-osteopontin antibody. The anti-osteopontin antibody can be a murine antibody, a humanized murine antibody, or a human antibody. Non-limiting examples of suitable anti-osteopontin antibodies include: C2K1, a chimeric antibody which specifically recognizes the human osteopontin epitope, SVVYGLR (SEQ ID NO:7); ASK8007 (Astellas Pharma Inc.); AOM1 (Pfzer Inc.), a fully human IgG2, and any other suitable anti-osteopontin antibody without limitation.

As another example, the osteopontin inhibitor can be a short hairpin RNA (shRNA) or a short interfering RNA (siRNA) targeting osteopontin.

Methods for preparing an osteopontin inhibitor (e.g., an agent capable of inhibiting osteopontin) can comprise the construction of a protein/Ab scaffold containing the osteopontin receptor as a neutralizing agent; developing inhibitors of the osteopontin receptor “down-stream”; or developing inhibitors of the osteopontin production “up-stream”.

Inhibiting osteopontin can be performed by genetically modifying osteopontin production in a subject or genetically modifying a subject to reduce or prevent expression of the osteopontin gene, such as through the use of CRISPR-Cas9 or analogous technologies, wherein, such modification reduces or prevents osteopontin production.

Molecular Engineering

The following definitions and methods are provided to better define the present disclosure and to guide those of ordinary skill in the art in the practice of the present disclosure. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule.

Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6(log 10[Na+])+0.41(fraction G/C content)−0.63(% formamide)−(600/l). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | GAPILV |
| Polar-uncharged | CSTMNQ |
| Polar-charged | DEKR |
| Aromatic | HFWY |
| Other | NQDE |

Conservative Substitutions II

| Side Chain Characteristic | Amino Acid |
|---|---|
| Non-polar (hydrophobic) | |
| A. Aliphatic: | ALIVP |
| B. Aromatic: | FW |
| C. Sulfur-containing: | M |
| D. Borderline: | G |
| Uncharged-polar | |
| A. Hydroxyl: | STY |
| B. Amides: | NQ |
| C. Sulfhydryl: | C |
| D. Borderline: | G |
| Positively Charged (Basic): | KRH |
| Negatively Charged (Acidic): | DE |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, osteopontin signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of osteopontin production by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)20NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for the osteopontin inhibitor to target distal and proximal tubule cells of an acutely injured kidney.

For example, the methods as described herein can comprise a method for altering a target polynucleotide sequence in a cell comprising contacting the polynucleotide sequence with a clustered regularly interspaced short palindromic repeats-associated (Cas) protein.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

Therapeutic Methods

Also provided is a process of treating or preventing an acute kidney injury-induced acute lung injury (AKI-ALI) by administration of a therapeutically effective amount of an osteopontin inhibitor.

Methods described herein are generally performed on a subject in need thereof. A subject in need of the therapeutic methods described herein can be a subject having, diagnosed with, suspected of having, or at risk for developing AKI-ALI. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of the osteopontin inhibitor is, for example, an amount that would cause the desired therapeutic effect in a subject while minimizing undesired side effects. In various embodiments, an effective amount of the osteopontin inhibitor described herein can substantially inhibit AKI-ALI, slow the progress of AKI-ALI, or limit the development of AKI-ALI.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of the osteopontin inhibitor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit AKI-ALI.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of the osteopontin inhibitor can occur as a single event or over a time course of treatment. For example, the osteopontin inhibitor can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for AKI-ALI.

The osteopontin inhibitor can be administered simultaneously or sequentially with another agent, such as an antibiotic, an anti-inflammatory, or another agent. For example, the osteopontin inhibitor can be administered simultaneously with another agent, such as an antibiotic or an anti-inflammatory. Simultaneous administration can occur through the administration of separate compositions, each containing one or more of the osteopontin inhibitor, an antibiotic, an anti-inflammatory, or another agent. Simultaneous administration can occur through the administration of one composition containing two or more of the osteopontin inhibitor, an antibiotic, an anti-inflammatory, or another agent. The osteopontin inhibitor can be administered sequentially with an antibiotic, an anti-inflammatory, or another agent. For example, the osteopontin inhibitor can be administered before or after the administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to the osteopontin inhibitor. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

The following examples make use of the methods described in detail below.

Animals

For all studies, adult (8 to 12 weeks old) male mice were used in accordance with the animal care and use protocol approved by the Institutional Animal Care and Use Committee of Washington University School of Medicine. C57BL/6J (B6) (JAX stock no. 000664) and B6.12956(Cg)-Spp1tm1Blh/J (OPN−/−) (JAX stock no. 004936) were purchased from the Jackson Laboratory.

Surgeries

Bilateral renal ischemia for 16 or 20 min (mild AKI) at 37° C. was induced in both kidneys using the flank approach as previously re-ported by cross-clamping both renal pedicles. Sham operations were performed with exposure of both kidneys but without induction of ischemia. Syngeneic wild-type (WT) (B6 to B6) or syngeneic KO (OPN−/− to B6) kidney transplants were performed as follows: Anesthesia was induced by a mixture of ketamine (80 to 100 mg/kg) and xylazine HCl (8 to 12 mg/kg) intraperitoneally and maintained with 1 to 2% isoflurane gas, as required. Briefly, B6 or OPN−/− donor kidneys were implanted into the abdominal cavity of B6 recipient mice, where the donor suprarenal aorta and renal vein were anastomosed to the recipient infrarenal aorta and inferior vena cava, respectively. The ureter was reconstructed by direct ureter to bladder insertion. Donor kidneys were subjected to 20-min warm ischemia after procurement before they were maintained on ice for implantation, and cold ischemic times were less than 40 min. Sham-operated mice underwent the same surgical procedure except for the transplant.

Single-Cell Preparation for RNA Sequencing

Kidneys were minced into small pieces (<1 mm³) and incubated in tissue dissociation buffer [Liberase™ (1 mg/ml), hyaluronidase (0.7 mg/ml), and deoxyribonuclease (80 U/ml) in phosphate-buffered saline (PBS)] for 30 min in 37° C. Single cells were released from the digested tissue by pipetting 10 times, and the cell suspension was filtered through a 70-mm sieve (Falcon). Fetal bovine serum (10%) was added to stop the enzymatic reaction. Cells were collected by centrifugation (300 g at 4° C. for 5 min) and resuspended in red blood cell (RBC) lysis buffer [155 mM NH4Cl, 10 mM KHCO3, and 1 mM EDTA (pH 7.3)] for 1 min at room temperature. After washing in PBS, cells were used fresh for analysis by scRNAseq.

Single-Cell RNA Sequencing

scRNAseq analysis of four pooled sham or four pooled AKI samples, for both kidney and lung (sham kidney/AKI kidney and sham lung/AKI-ALI lung), was performed. Briefly, cells were stained with propidium iodide, and live cells were sorted using FACSAria III (BD Biosciences). Libraries were prepared using the Chromium Single Cell 5′ Library Kit v2 and Chromium instrument (10× Genomics, Pleasanton, Calif.). Full-length cDNA was amplified, and libraries were submitted to Genome Technology Access Center of Washington University in St. Louis for sequencing at a depth of 50,000 reads. All processing steps were performed using Seurat v3. Quality control was first performed on each library to find appropriate filtering thresholds for each. Expression matrices for each sample were loaded into R as Seurat objects, retaining only cells that have more than 200 and less than 3200 genes. Poor quality cells with >10% mitochondrial genes were removed. Any gene not expressed in at least three cells was removed. sctransform was used for normalization scaling and variance stabilization (https://github.com/ChristophH/sctransform). This was done to reduce bias introduced by technical variation, sequencing depth, and capture efficiency. An identifier for sham kidney, AKI kidney, sham lung, and AKI-ALI lung was assigned to tell them apart. Integration of kidney and lung single-cell data was done using the harmony pack-age (https://github.com/immunogenomics/harmony) to control for batch effects when integrating data from different samples. After quality control and integration, 13,882 kidney cells and 15,167 lung cells were further analyzed. 15 clusters in the kidney and 20 clusters in the lung were identified. Cell clustering was visualized using uniform manifold approximation and projection (UMAP). To assign cluster identities, a list of lung and kidney cell types and their currently established markers were first compiled and the expression of those markers and additional known canonical markers were assessed using the FindAllMarkers( ) function in Seurat.

L-R Pairing Analysis

To infer cell-to-cell communication between kidney and lung cell types from scRNAseq or snRNAseq data, L-R pairing analysis was performed using CellPhoneDB (cellphonedb.org). First, all datasets from kidney [sham kidney and AKI kidney (4, 12, and 24 hours)] and lung (sham lung and AKI-ALI lung) were integrated using reciprocal principal components analysis as implemented in Seurat. The 4- and 12-hour AKI kidney snRNAseq data were from published data. CellPhoneDB contains a highly curated set of human protein-protein interactions and protein complexes; thus, mouse genes were mapped to their high-confidence human one-to-one ortholog using homology mappings from Ensembl. CellPhoneDB statistical analysis was performed with default settings between all kidney and lung cell populations, conditions, and time points simultaneously to increase statistical power. Last, only co-expressed pairs with a ligand expressed in a kidney cell population and its cognate receptor expressed in a lung cell population with significant cell type-specific coexpression were considered as compared to randomly shuffled cells (P<0.01). CellPhoneDB calculates an empirical P value of significance, with higher P values indicating higher significance.

Histology, Immunofluorescence Staining, and Quantification

Mice were anesthetized by ketamine cocktail [ketamine (20 mg/ml) and xylazine (2 mg/ml) in 0.9% sodium chloride solution] and then perfused with PBS. Lungs were harvested at the indicated times. Four lobes of the right lung were cut and divided. For histology, two lobes (superior and middle lobes) were inserted into 4% para-formaldehyde, located inside a 10-ml syringe, and inflated by creating pressure with the syringe plunger after locking the syringe. After inflation, the lobes were fixed in 4% paraformaldehyde overnight at 4° C. and then processed and embedded in paraffin. Sections (4 mm in thickness) were stained with hematoxylin and eosin. Alveolar wall thickness was measured as previously described using ImageJ. For immunofluorescence, one lobe (inferior lobe) of the right lung was placed into PBS, located inside a 10-ml syringe, inflated by creating pressure with the syringe plunger after locking the syringe. Fresh frozen lung sections (7 mm) were fixed in 4% paraformaldehyde, permeabilized in 0.1% Triton X-100 for 3 min, and blocked for 1 hour with 10% normal goat serum supplemented with 1% bovine serum albumin (BSA) in PBS. Sections were incubated with primary antibodies in blocking solution overnight at 4° C. The primary antibodies used were rat anti-mouse Ly6G/Ly6C (clone RB6-8C5, 14-5931, eBioscience; 1:100), rabbit anti-CD68 (ab125212, Abcam; 1:200), rat anti-mouse CD31 (550274, BD Pharmingen; 1:200), rabbit anti-ZO1 (ab221547, Abcam; 1:500), rat anti-nidogen (sc-33706, Santa Cruz Biotechnology; 1:200), rat anti-mouse CD44 (550538, BD Pharmingen; 1:200), and goat anti-mouse OPN (AF808, R&D Systems; 1:100). After extensive washes with PBS, fluorescently conjugated secondary antibodies were applied at 1:300 dilution for 1 hour at room temperature. The secondary antibodies used were as follows: Alexa Fluor 594 goat anti-rabbit (A-11037), Alexa Fluor 488 goat anti-rat (A-11006), Alexa Fluor 594 donkey anti-goat (A-11058), Alexa Fluor 647 donkey anti-rabbit (711-605-152), and Alexa Fluor 488 donkey anti-rat (712-545-153). For quantitative analysis, six representative areas that were captured with a Nikon Eclipse E800 microscope at a 200× magnification were selected.

Electron Microscopy

For transmission electron microscopy, small pieces (1- to 2-mm cubes) of lung tissue were fixed with 2% paraformaldehyde and 2% glutaraldehyde in 0.1 M sodium cacodylate buffer (pH 7.4). Embed-ding and sectioning were performed by the Washington University Center for Cellular Imaging. To assess the integrity of endothelial cell-cell junction, junction length was measured from digital images using ImageJ software expressed as a relation of electron dense cortical protein complex area to the total length of cell-cell contact between endothelial cells as previously described.

Evans Blue Injection

Mice were injected retro-orbitally with Evans blue dye (30 mg/ml; Sigma-Aldrich) in PBS (50 mg/g of body weight). After 20 min, mice were anesthetized and perfused via the left ventricle with 40 ml of PBS, and lungs were removed and additionally rinsed with PBS. The left lung was cut in half, and each half was weighed. Evans blue dye was extracted from one-half of the lung by incubation with 200 ml of formamide (56° C. for 24 hours), and the concentration of Evans blue was estimated by spectrophotometer (620 nm). The other half lung was dried in an incubator set at 65° C. The dry weight was obtained after 48 hours of incubation, and the ratio of wet-to-dry weight was calculated. The resulting unit of Evans blue plasma extravasation was optical density at 620 nm (OD620) per gram of dry weight.

OPN-Conjugation, Injection, and Detection

Recombinant mouse OPN (441-OP, R&D Systems) was conjugated to Alexa Fluor 555 (A30007, Molecular Probes) according to the manufacturer. Briefly, OPN protein was reconstituted at 1 mg/ml in sterile PBS. One vial of Alexa Fluor 555 succinimidyl ester was dis-solved in 10 ml of distilled H2O. The reaction mixture containing 25 ml of recombinant OPN, 1.66 ml of Alexa Fluor 555, and 2.5 ml of 1 M sodium bicarbonate was incubated for 15 min at room temperature. After purification, the concentration of the protein was read at 280 and 555 nm using NanoDrop ND-2000C spectrophotometer (Thermo Fisher Scientific). Alexa Fluor 555-conjugated OPN was intravenously injected into mice after 20 min of bilateral renal ischemia at reperfusion or in sham mice at an equivalent matching time point. Mice were euthanized 30 min or 1 hour later, and mice were anesthetized and perfused via the left ventricle with 40 ml of PBS, before lungs were removed and additionally rinsed with PBS. Lungs were then processed to frozen sections and assessed for deposition of OPN into lung tissue by confocal microscopy.

Human Samples

Deidentified human samples of patients with AKI without multiorgan failure, of patients with CKD or of healthy controls were provided by the Kidney Translational Research Core at Washington University in St. Louis. Cause of AKI was as follows: eight acute tubular necrosis (ATN), one oxalate crystal ATN, one AKI of obstructive etiology, one contrast nephropathy, one ureterovesical junction stone AKI, one AKI with HIV, and one AKI with malignant hypertension. Patients did not have clinical evidence of other organ failure based on clinical data review and serum biochemistries.

Quantitative Reverse Transcription Polymerase Chain Reaction

Total RNA was isolated from mouse kidneys and lungs using the Direct-zol RNA MiniPrep Plus Kit (catalog no. R2072) following the manufacturers' instructions. Total RNA was reverse transcribed using the QuantiTect RT Kit (QIAGEN) and real-time polymerase chain reaction (PCR) was performed with Fast SYBR Green (QIAGEN). Primer sequences are provided in Table 1. Glyceraldehyde-3-phosphate dehydrogenase (Gapdh) was used as the housekeeping gene. Data were analyzed using the DDCt method.

Enzyme-Linked Immunosorbent Assay

Human OPN, mouse OPN, and mouse KIM-1 were measured in human or mouse serum samples using enzyme-linked immunosorbent assay (ELISA) kits (DY1433, DY441, and DY1817, respectively, all from R&D Systems) as per the manufacturer's instructions. Serum dilutions for ELISAs were as follows: mouse KIM-1 (1:10 dilution); mouse OPN: sham (1:2000 dilution), injured (1:4000 dilution), sham transplant (1:1000 dilution), and WT kidney transplant and OPN-KO kidney transplant (1:2000 dilution); human serum OPN: healthy control (1:500 dilution), patients with AKI (1:1000 dilution), and patients with CKD (1:500).

Mass Cytometry CyTOF

Single-cell preparations were analyzed by mass cytometry as previously described. Briefly, cells were labeled using a previously validated and titrated antibody cocktail for surface markers (all anti bodies conjugated by the manufacturer; Fluidigm) diluted in Fluidigm MaxPar Cell Staining Buffer (CSB) (1 hour at 4° C.). After two washes in CSB, cells were fixed in 2% paraformaldehyde for 20 min at room temperature, washed, stained with MaxPar Intercalator-IR (Fluidigm), and filtered into cell strainer cap tubes. Data were then acquired on a CyTOF2/Helios instrument (Fluidigm) and analyzed with the CytoBank software using our recently described gating strategy.

TABLE 1
Primers.

| Target | Primer sequence forward | Primer sequence reverse |
|---|---|---|
| gene | (5′→3′) | (5′→3′) |
| Gapdh | ACCACAGTCCATGCCATCAC | TCCACCACCCTGTTGCTGTA |
| | (SEQ ID NO: 1) | (SEQ ID NO: 4) |
| Havcr1 | AAACCAGAGATTCCCACACG | GTCGTGGGTCTTCCTGTAGC |
| (Kim-1) | (SEQ ID NO: 2) | (SEQ ID NO: 5) |
| Spp1 | GGATGAATCTGACGAATCTC | GCATCAGGATACTGTTCATC |
| | (SEQ ID NO: 3) | (SEQ ID NO: 6) |

Renal Function

Serum creatinine was assessed by a liquid chromatography-mass spectrometry-based assay at the O'Brien Core Center for Acute Kidney Injury Research (University of Alabama School of Medicine, Birmingham, Ala., USA). BUN levels were measured using the DiUR100 kit (Thermo Fisher Scientific) according to the manufacturer's instructions.

Statistical Analysis

Statistical analyses were carried out using GraphPad Prism. Two-tailed, unpaired t tests, one-sample t tests, one-way analysis of variance (ANOVA), and two-way ANOVA were used to determine statistical significance in quantification. All results were expressed as means±SD, and P<0.05 was considered as statistically significant.

BAL Analysis

BAL fluid was obtained by instilling saline into the lungs through a tracheal cannula using a volume equal to 80% of lung vital capacity (3× with 0.5 ml of 0.9% NaCl) for a total of 1.5 ml. Total BAL fluid recovery was approximately 90% of the instilled volume. The BAL fluid was centrifuged (400 g for 10 min at 4° C.), and the cell pellet was resuspended in PBS.

Protein and Albumin Concentration

Total protein concentrations in the cell-free BAL supernatant were determined using the Pierce BCA Protein Assay Kit (WJ334006, Thermo Fisher Scientific, Berkeley, Mo.). Albumin levels were determined with an ELISA quantification kit (W90-13, Bethyl Laboratories, Montgomery, Tex.) according to the manufacturers' specifications.

Flow Cytometry (Fluorescence-Activated Cell Sorting) Analysis of BAL Cells

The cell pellet was resuspended in 0.2 ml of RBC Lysis buffer and incubated for 2 min at room temperature. Samples were blocked with Fc block (#101301, BioLegend) for 20 min before labeling for 30 min at 4° C. with antibodies and washed in fluorescence-activated cell sorting (FACS) buffer (0.5% BSA, 2 mM BSA, and 0.01% NaN3). The following antibodies, coupled to a fluorophore, were used to identify the different cell types: CD45 (#103140, BioLegend), Cd11b (#565976, BD Biosciences), Ly6G (#127627, BioLegend), CD64 (#139315, BioLegend), MERTK (MER proto-oncogene, tyrosine kinase) (#151505, BioLegend), and SiglecF (#740956, BD Biosciences). Dead cells were excluded using LIVE/DEAD Fixable Aqua stain (#L34957, Thermo Fisher Scientific). Compensation was performed at the time of sample acquisition, and flow cytometry results were analyzed using FlowJo version 10.8.1 (FlowJo LLC). All experiments were performed using BD LSRFortessa X-20. Antibody selection and manual gating strategies were defined on the basis of previous literature.

### Example 1: Ligand-Receptor Pairing Analysis Across Organs During Multiorgan Failure Identifies Kidney-Released Circulating Osteopontin as a Mediator of Lung Vascular Leakage, Inflammation and Respiratory Failure

To characterize the role of osteopontin in acute kidney injury-induced acute lung injury (AKI-ALI), the following experiments were conducted.

Ischemic AKI Causes Severe ALI

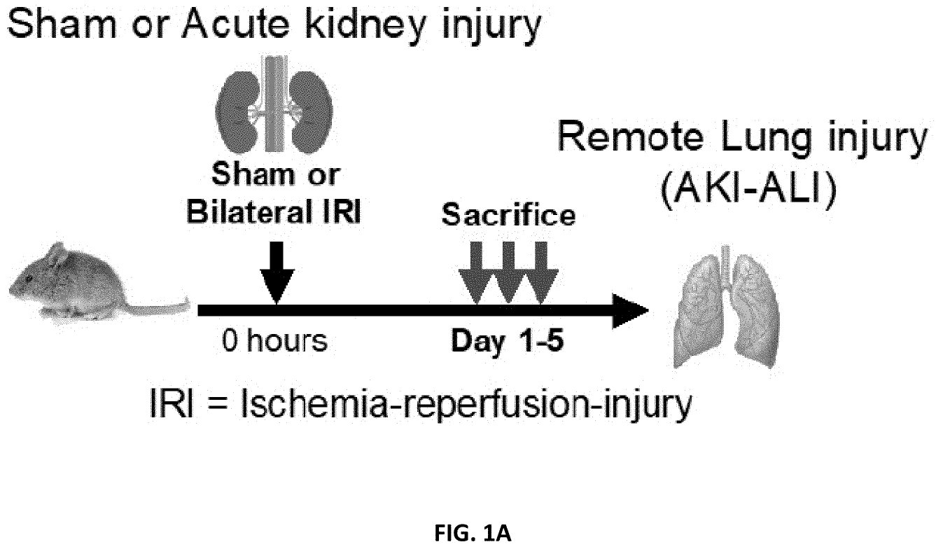

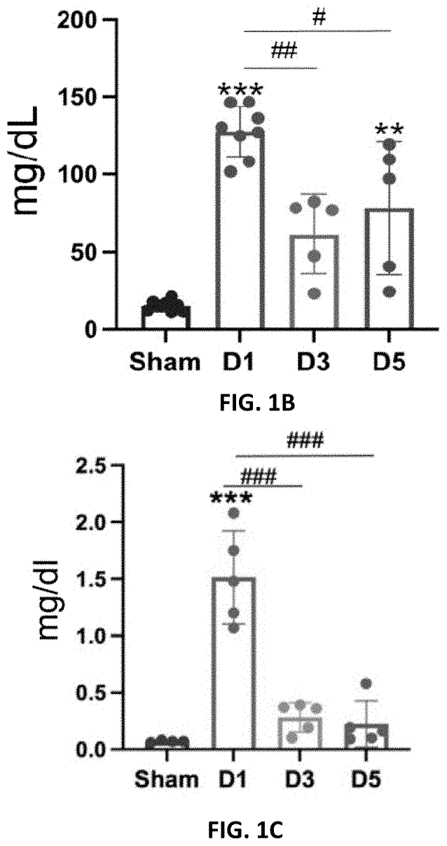

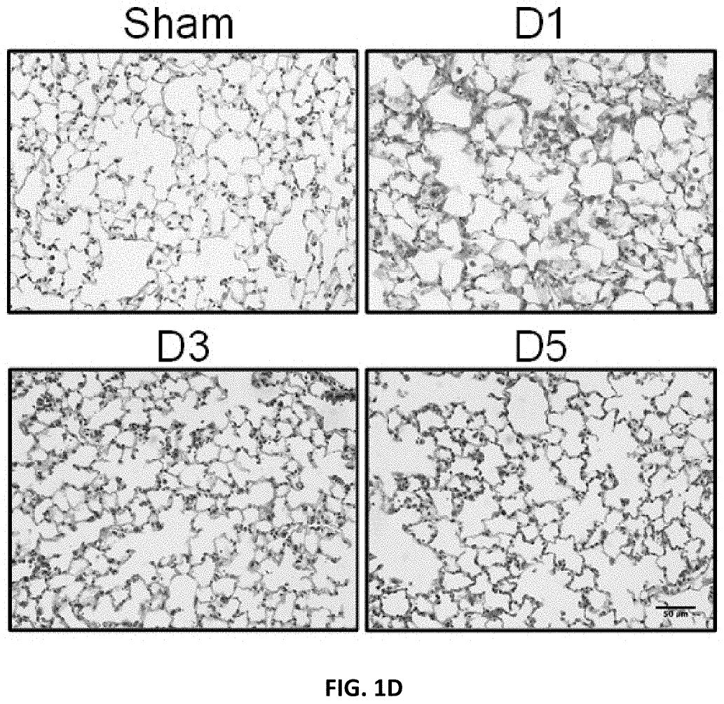

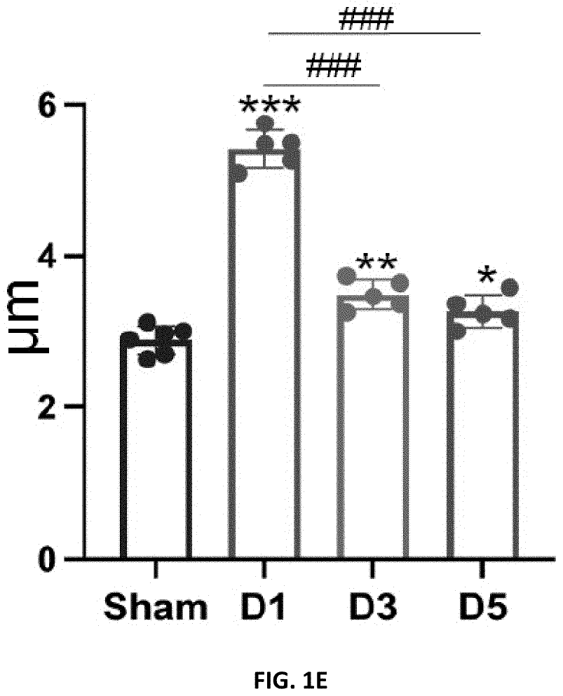

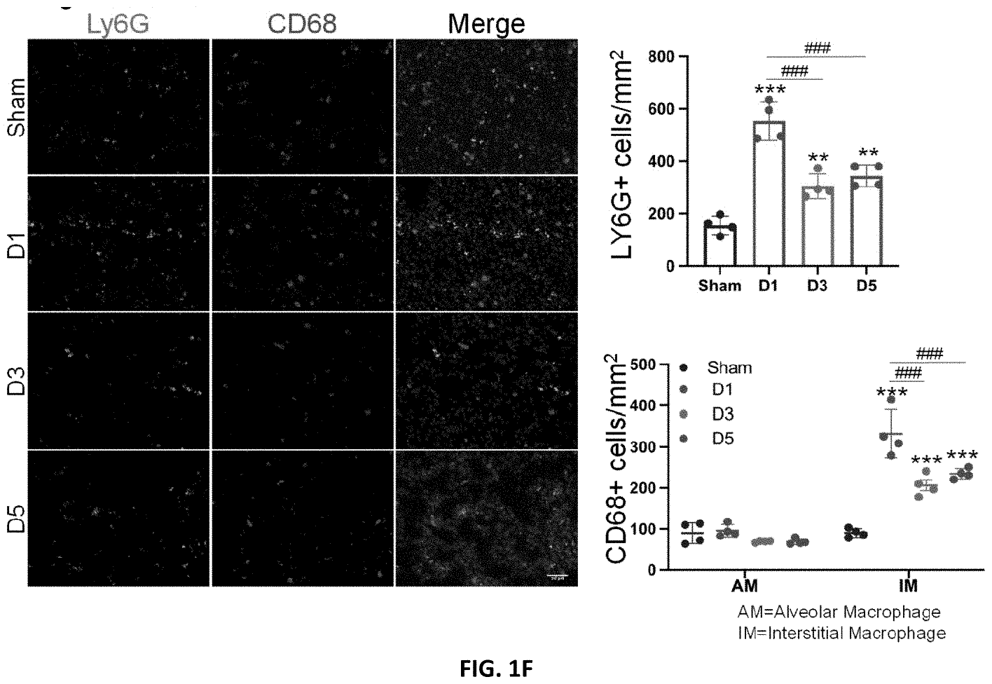

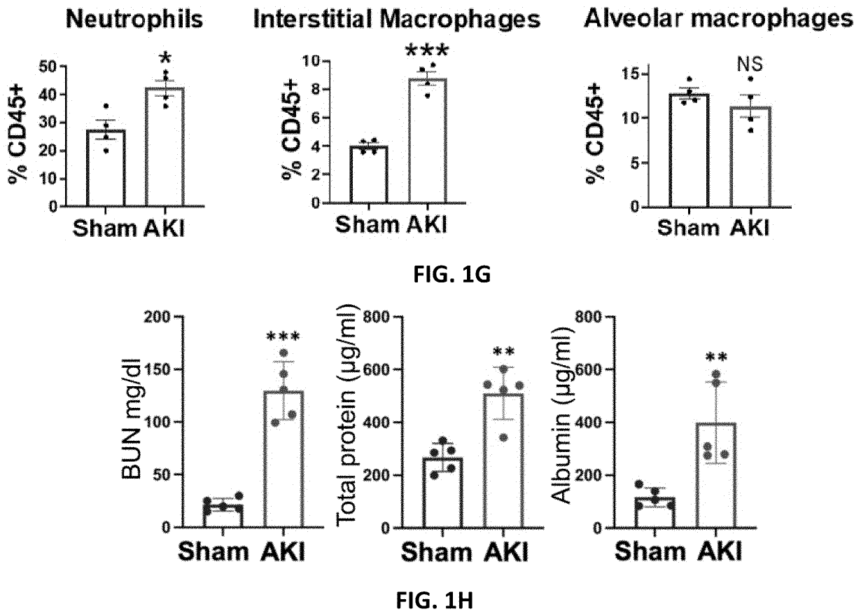

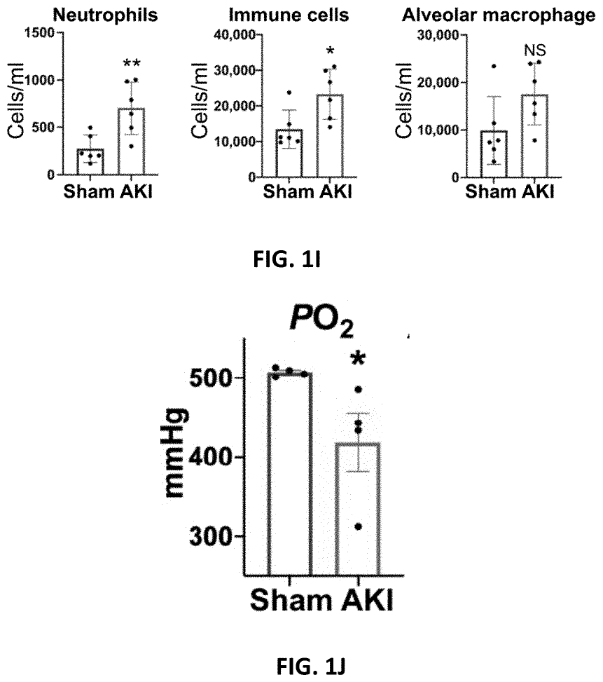

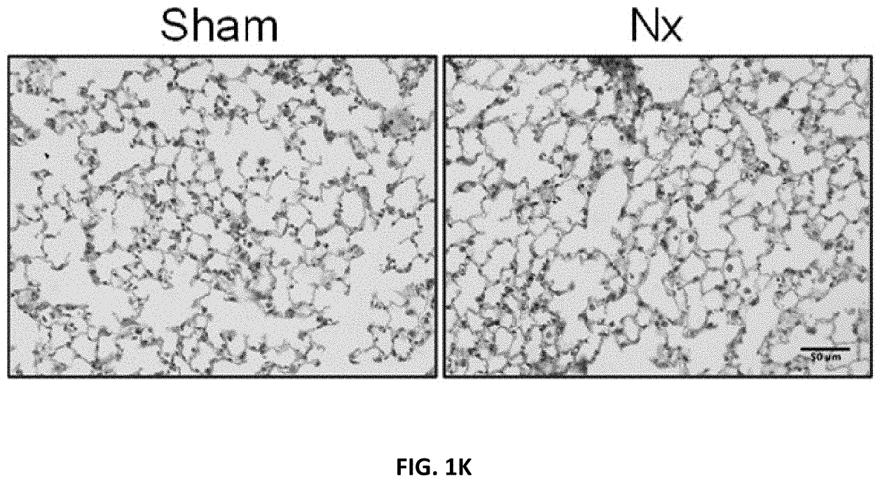

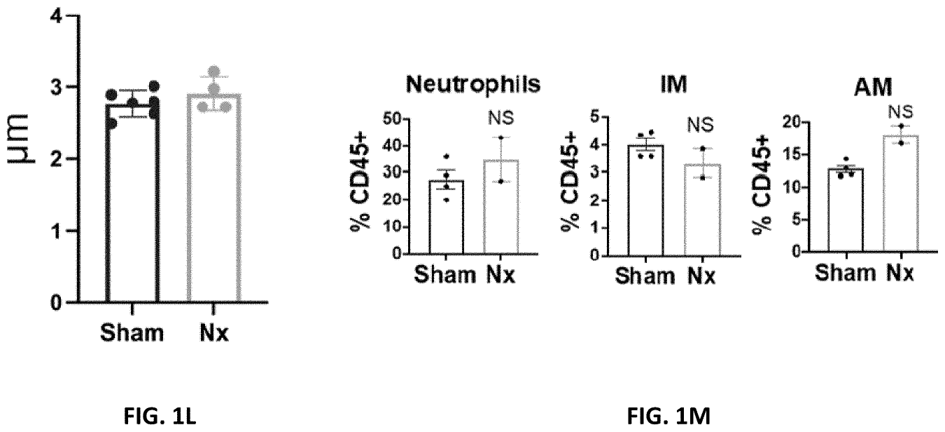

Aiming at identifying communication between an injured tissue and other organs, a model of multiorgan failure was established. The kidney was injured and remote effects on the lung was studied. C57BL/6 mice were subjected to bilateral renal ischemia-reperfusion injury (IRI) and analyzed remote ALI (AKI-ALI) (FIG. 1A, experimental scheme). Kidney injury caused highly elevated blood urea nitrogen (BUN) levels on day 1 after AKI, indicating severe kidney failure, with slow improvement by about 50% over days 3 to 5 (FIG. 1B). Consistent with this, serum creatinine was significantly elevated on day 1 and remained elevated at lower levels over days 3 to 5 (FIG. 1C). Severe injury to lungs and inflammation (AKI-ALI) followed with the same kinetics, as determined by expansion of interstitial spaces, increased cellularity and interstitial edema (FIG. 1D). Alveolar wall thickness increased over sham levels by 75% on day 1, signaling impediment of oxygenation (FIG. 1E). Neutrophils and interstitial macrophages (IMs) accumulated in the lung, peaked on day 1 after AKI, and were still significantly elevated by day 5. IMs that accumulated in lungs after AKI are small and CD68low. Alveolar macrophages (CD68high) did not change in numbers (FIG. 1F). These results were confirmed by mass cytometry [cytometry by time of flight (CyTOF)] of lung single-cell preparations from sham and AKI mice (FIG. 1G). Analysis of lung bronchoalveolar lavage (BAL) fluid on day 1 after AKI as compared to BAL fluid from sham controls showed significantly increased total protein and albumin concentration (FIG. 1H), as well as increased total immune cells and neutrophils; alveolar macrophage numbers were similar to sham control (FIG. 1I). These BAL findings are consistent with substantial AKI-ALI. The functional consequence of these changes was a significant impairment in oxygen exchange on day 1 after AKI (FIG. 1J). The mouse data thus closely resemble what can be observed in patients with multi-organ failure after AKI.

To exclude that the observed AKI-ALI phenotype is simply the result of accumulation of toxic waste products or failure to excrete an existing potentially detrimental molecule because of lack of glomerular filtration, mice for AKI-ALI were examined on day 1 after simple bilateral nephrectomy, without reperfusion injury. Sham-operated mice or animals that underwent bilateral nephrectomies showed no differences in terms of lung interstitial spaces and cellularity (FIG. 1K), alveolar wall thickness (FIG. 1L), or lung immune cell numbers as assessed by CyTOF on day 1 after nephrectomy (FIG. 1M). These findings indicate that AKI-ALI in our model does not develop simply because of a failure to excrete a potentially detrimental molecule.

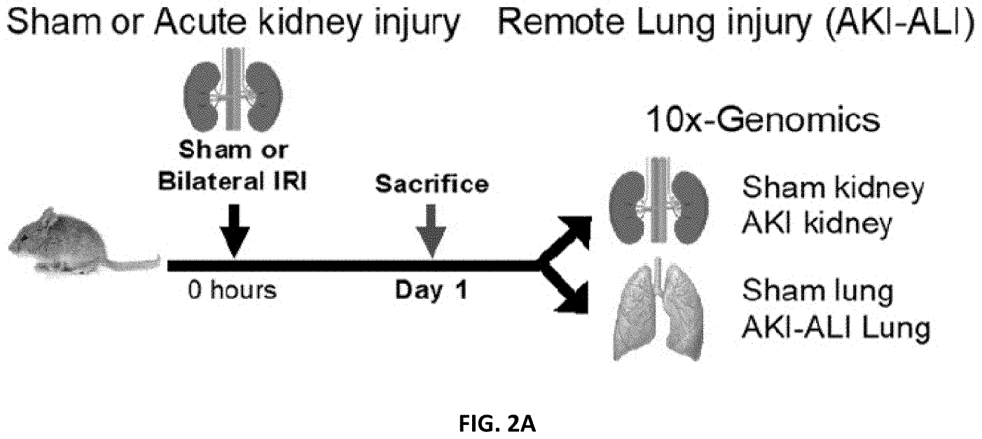

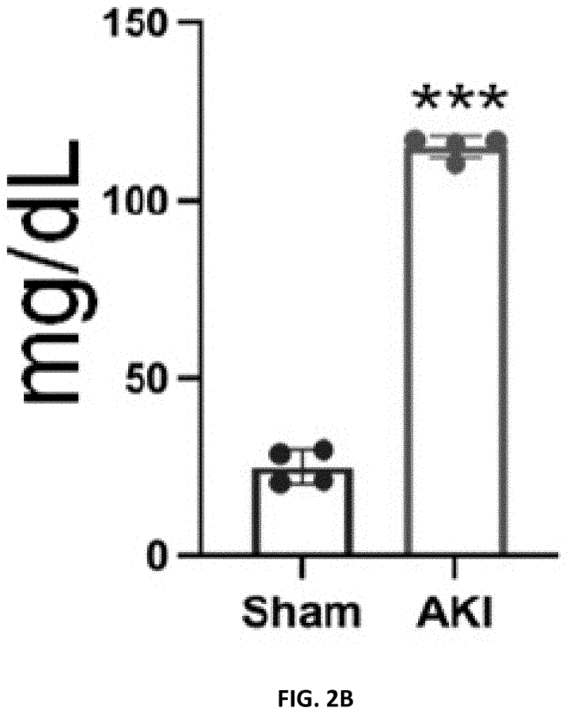

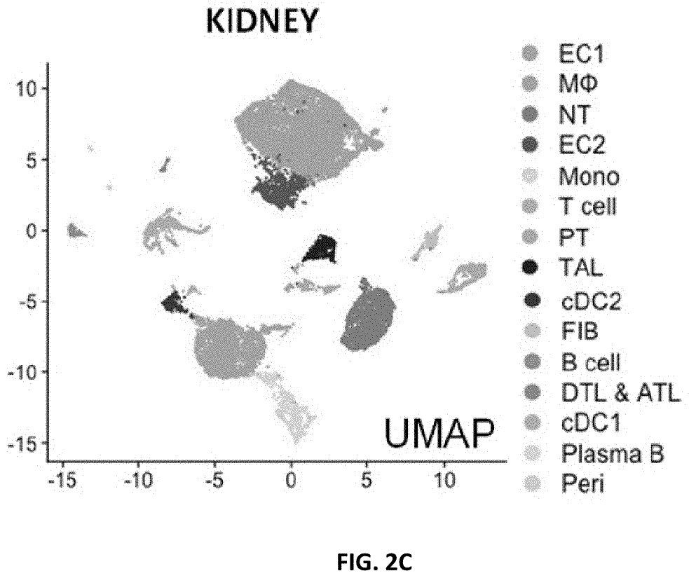

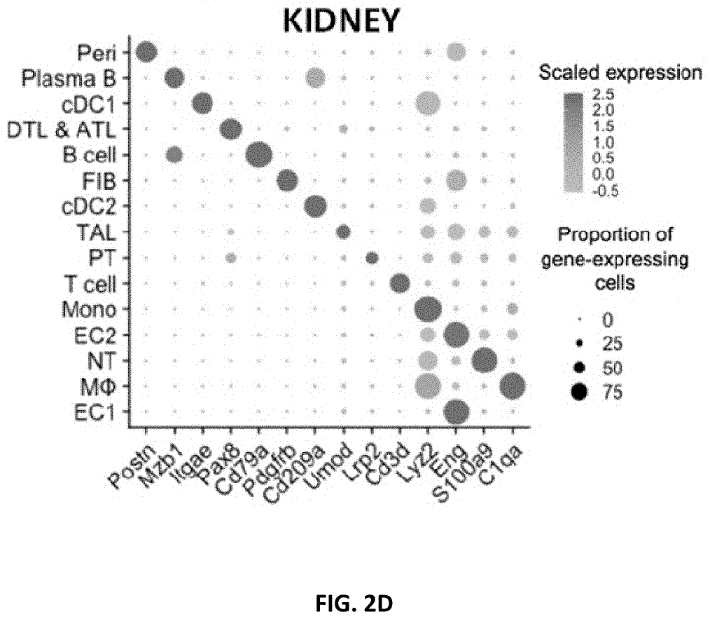

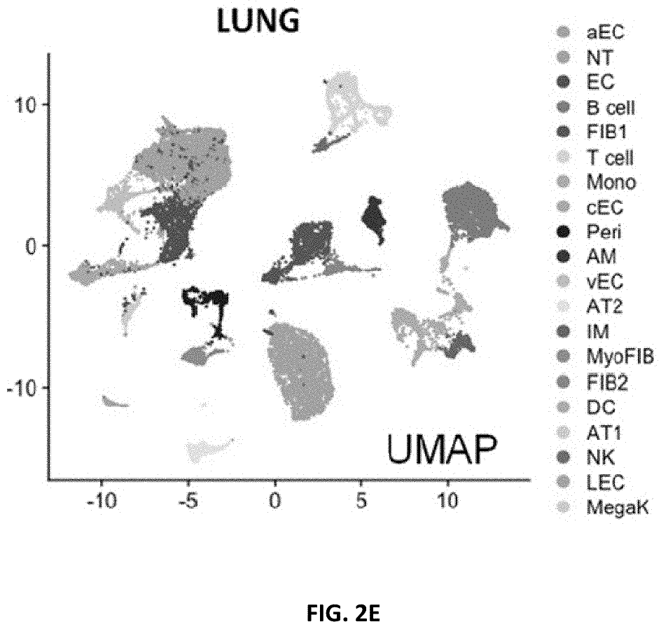

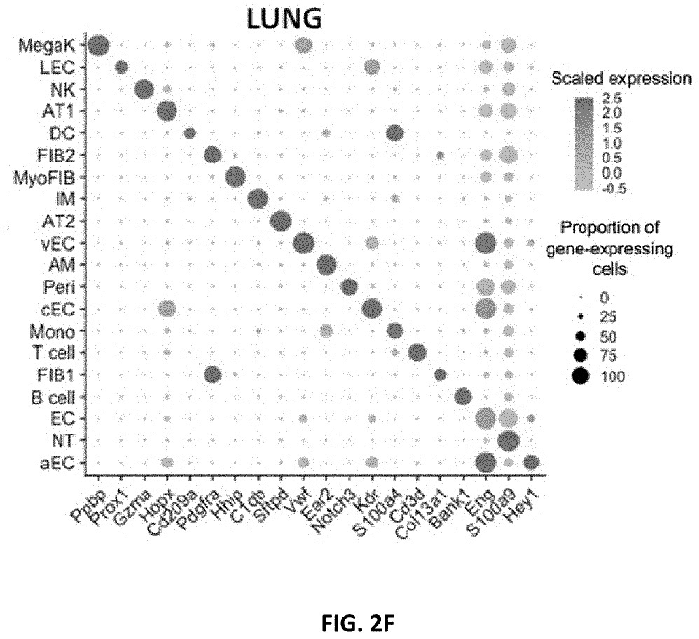

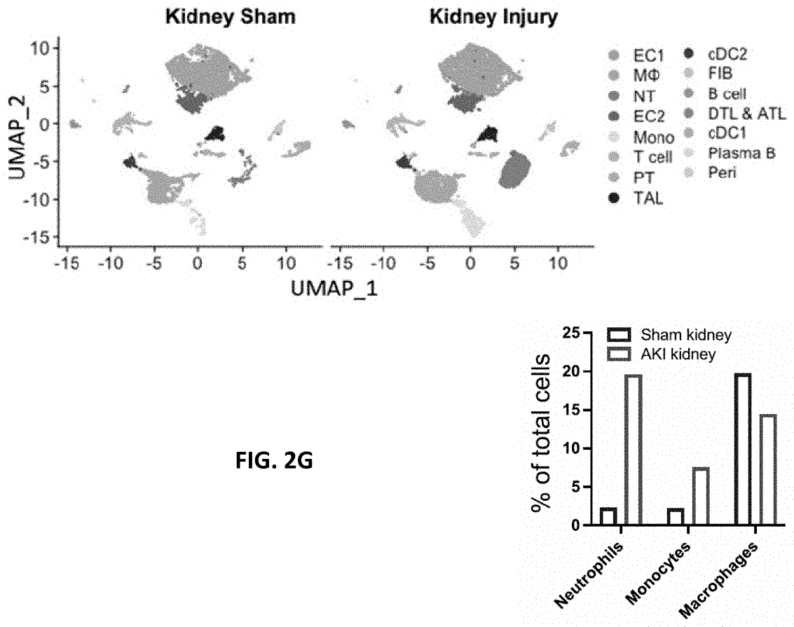

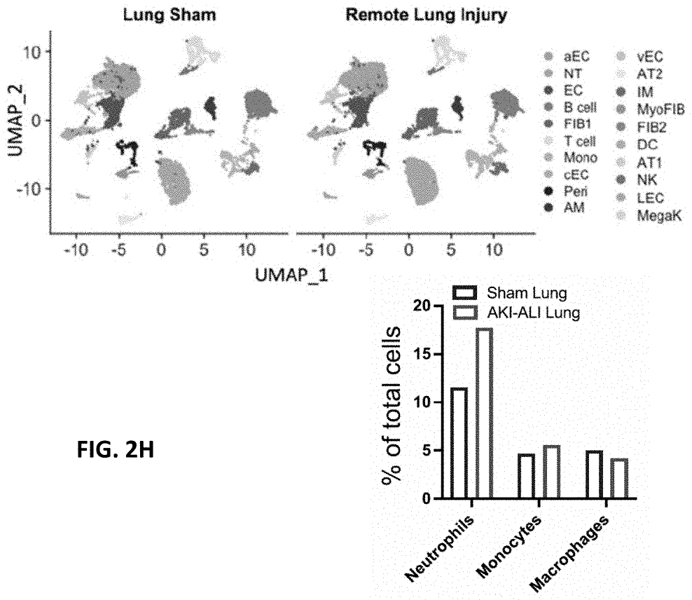

scRNAseq of kidney and lung in setting of AKI-ALI As an entrance into identifying relevant cellular changes after AKI, cellular gene expression profiles were examined by scRNAseq of kidneys and lungs isolated on day 1 after sham operation or bilateral renal IRI-induced AKI (FIG. 2A, experimental scheme). Serum BUN levels were significantly elevated in injured mice on day 1 after AKI (FIG. 2B), indicating severe AKI. Analysis of scRNAseq gene expression profiles revealed the presence of all known nonimmune and immune cell types of the kidney and lung. Seurat objects that combine the data for sham and injury, as well as dotplots identifying standard marker genes used to identify cell types, for either kidney (FIGS. 2C and 2D) or lung (FIGS. 2E and 2F), are shown. To monitor specific changes between sham and injury, scRNAseq data were replotted separately for sham and injury of the kidney or lung. On the basis of number of cells captured in each population (Seurat cluster), the injured kidney in comparison to sham showed easily detectable and expected dynamics in the immune cell compartment, with strong enrichment of neutrophils and monocytes in the injured sample (FIG. 2G). Similar but somewhat more subtle changes were detected in the same immune cells in the remotely injured lung. A strong increase in the number of neutrophils and a small increase in monocytes in remotely injured lung tissue were detected (FIG. 2H). Changes in lung IM numbers between sham and injury were less appreciable at this level of the scRNAseq analysis, although they were detected in large numbers (FIGS. 2G and H).

L-R Pairing Analysis Across Organs, Linking Ligands Expressed in the Kidney to Receptors Expressed in the Lung

To infer possible cell-cell communication events between kidneys and lungs, the machine learning algorithm CellPhoneDB was used to perform computational L-R pairing analysis across organs, with ligands located in the kidney and their cognate receptors in the lung (FIG. 3A). The CellPhoneDB L-R interaction database used here is unique in that, beyond classical ligand and receptor interactions, it also considers other interaction partners that may participate in signaling, such as co-receptors or other receptor-associated proteins. Only L-R interactions with the ligand expressed in a kidney cell type and its cognate receptor expressed in a lung cell type were considered. Notably, CellPhoneDB calculates an empirical P value, with higher P values indicating higher predicted significance of the L-R pairing.

The analysis was divided into L-R pairings from nonimmune cells or immune cell types in the kidney to nonimmune cells or immune cell types in the lung, either at baseline (sham) or after injury (AKI kidney or AKI-ALI lung). The top scoring L-R pairing detected at baseline and after injury was tubule cell-expressed OPN [or secreted phosphoprotein-1 (SPP1)] pairing with its receptor CD44 in lung nonimmune and immune cell types. The significance of these predicted interactions was P>2.5 at baseline, which significantly increased to P>4.5 after injury. At baseline, distal tubule cells represented the main source of OPN, but, by day 1 after AKI, proximal tubule cells were also predicted to significantly participate in OPN: CD44 signaling (FIGS. 3B and 3C, panels 1 and 2). CD44 has several other ligands, some of which were also detected in the L-R pairing analysis, such as hepatocyte growth factor (HGF):CD44 or fibro-blast growth factor 2 (FGF2):CD44, but these pairings were limited to kidney immune to lung immune cell pairings and of low to moderate significance that did not change with injury (FIG. 3D, panels 1 and 2). For kidney immune cells, the top scoring L-R pairings, however, with low difference in significance between sham and injured samples (P>2.5→3) included (i) kidney neutrophil IL1b connecting to b2-adrenergic receptor (IL1b:Adrb2) in lung immune or stromal cells and (ii) kidney T cell chemokine ligand 5 (CCLS) connecting to CCR1-5/atypical chemokine receptor 4 (Ackr4) receptors in lung immune or nonimmune cells (FIGS. 3D AND 3E, panels 1 and 2). The significance of the IL1b:Adrb2 pairing is unclear, as this pairing is predicted by CellPhoneDB on the basis of protein-protein interaction data curated from various experimental approaches, but it is not known whether this interaction produces a cellular signal. Whether T cell released CCLS or CCLS, in general, is involved in AKI-ALI is unknown.

The CellPhoneDB analysis was validated using two approaches. First, a previously published snRNAseq dataset was used that assessed kidney gene expression at early time points after AKI for our L-R pairing analysis. Their kidney snRNAseq dataset of 4 and 12 hours after AKI was linked to our scRNAseq data of the remotely injured AKI-ALI lung at day 1 after AKI. L-R pairing analysis identified OPN:CD44 as a top hit. Kidney distal tubule OPN pairing with CD44 receptor in lung immune and nonimmune cell types showed high significance at 4 hours after AKI. At 12 hours after AKI, OPN derived from several kidney cell types including distal tubule cells, endothelial cells, fibroblasts, macrophages, and T cells pairing with CD44 in lung immune or nonimmune cells represented the top scoring L-R pairing (FIGS. 5A and 5B). Second, a list of all secreted proteins identified in UniProt was used and a hand-curated list of additional secreted or metalloprotease released proteins of interest were added and their average expression in all cell types of the AKI kidney scRNAseq dataset were assessed. In this analysis, OPN also emerged as the top distal tubule secreted molecule after AKI, and none of the secreted proteins, which were not included in CellPhoneDB, showed a comparable increase.

OPN (SPP1), initially identified as a regulator of bone biomineralization and remodeling, is an immunoregulatory molecule expressed in a variety of cells, including stromal, epithelial, and immune cells. OPN is strongly chemotactic for immune cells, in particular, for neutrophils and macrophages and enhances T helper 1 inflammation. Taking these immunoregulatory features of OPN and our L-R pairing analysis and in silico validation into account, it was hypothesized that OPN might be a good candidate AKI-ALI mediator to study in our AKI-ALI model.

OPN is Up-Regulated in Kidneys but not in Lungs During AKI-ALI

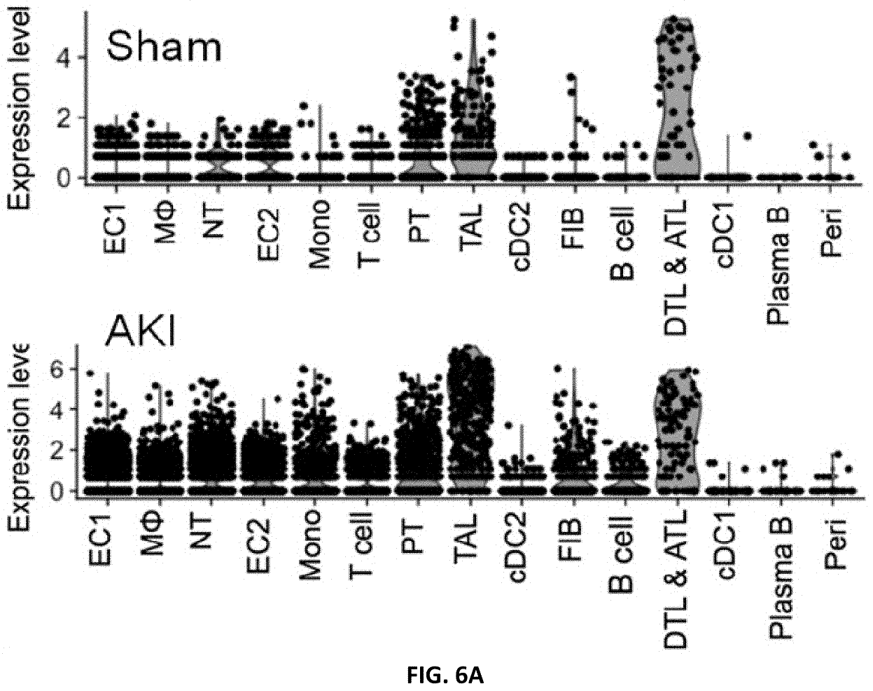

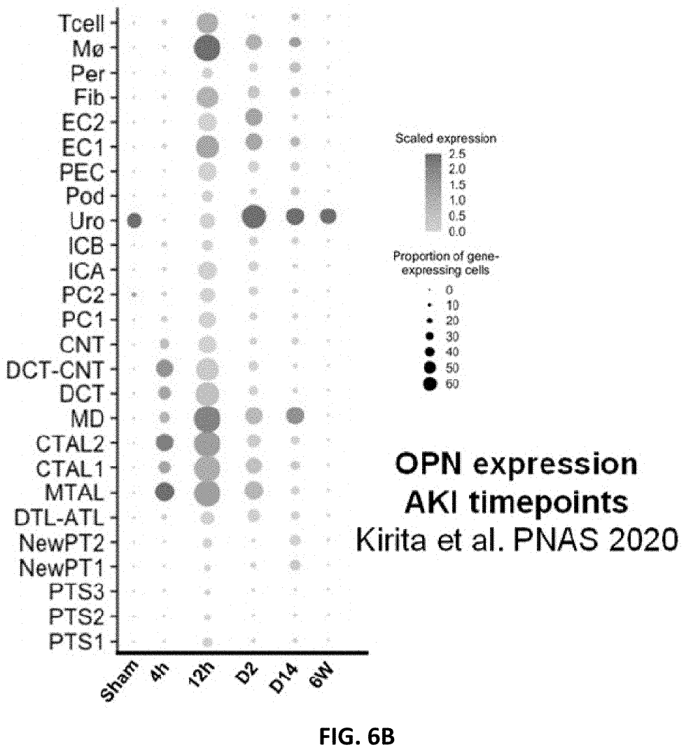

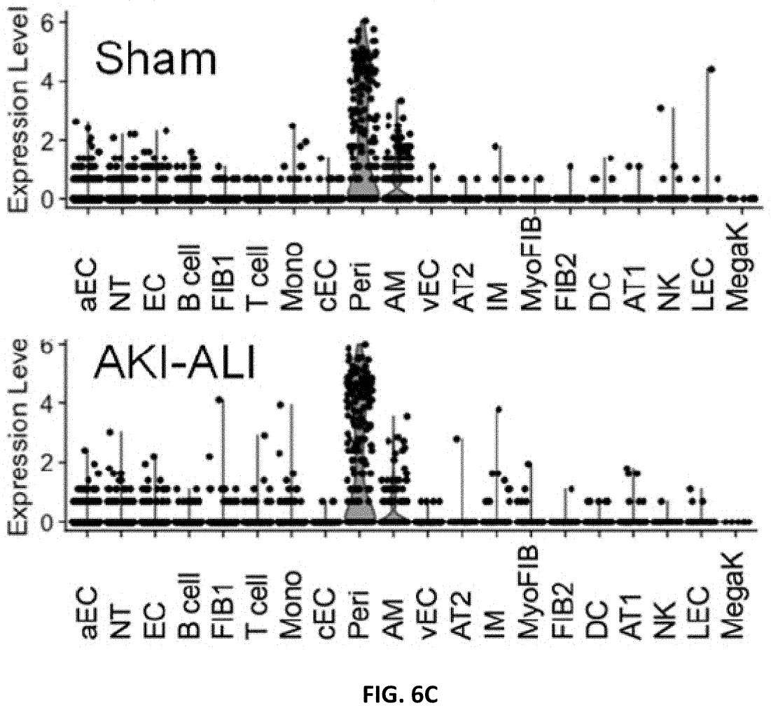

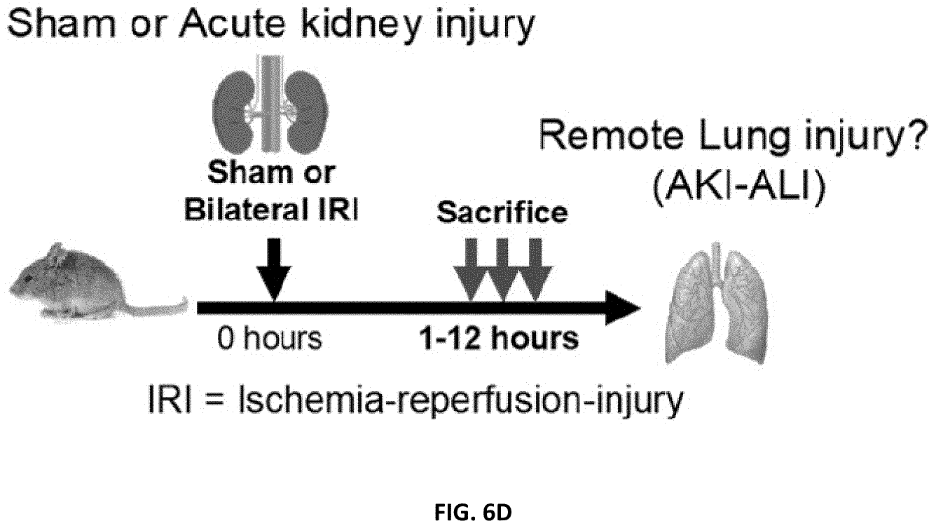

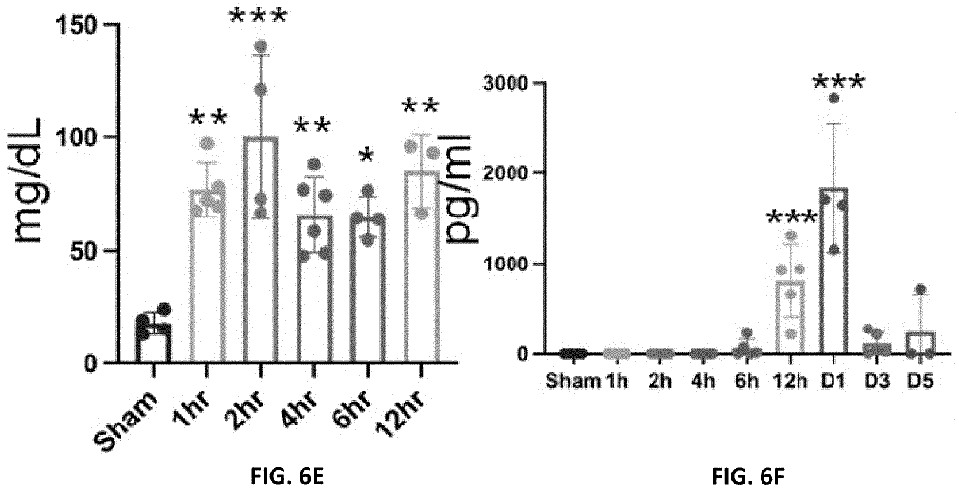

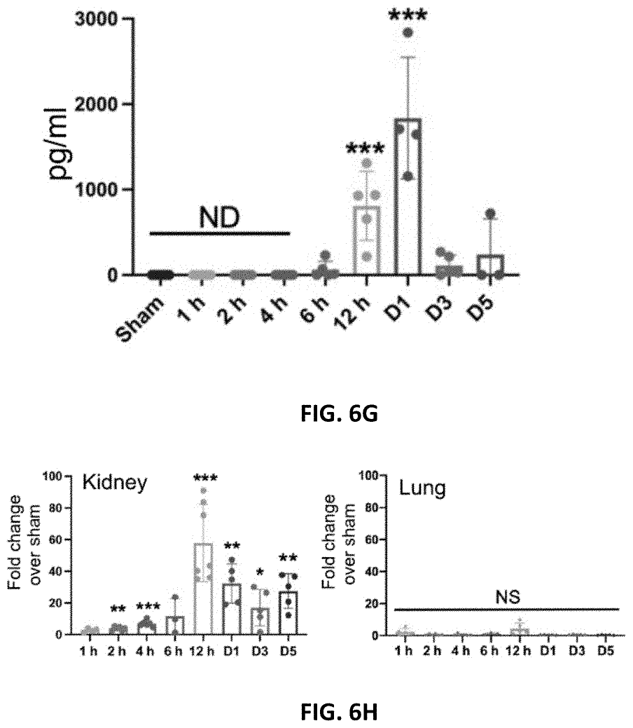

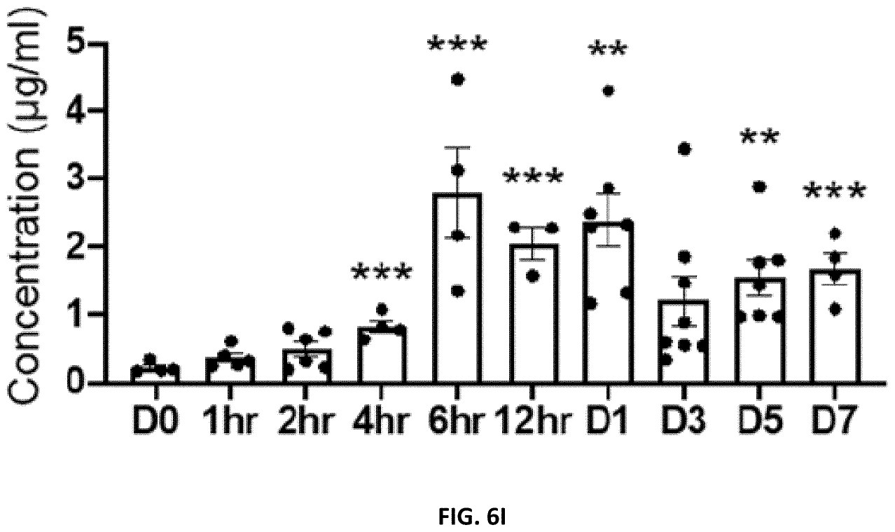

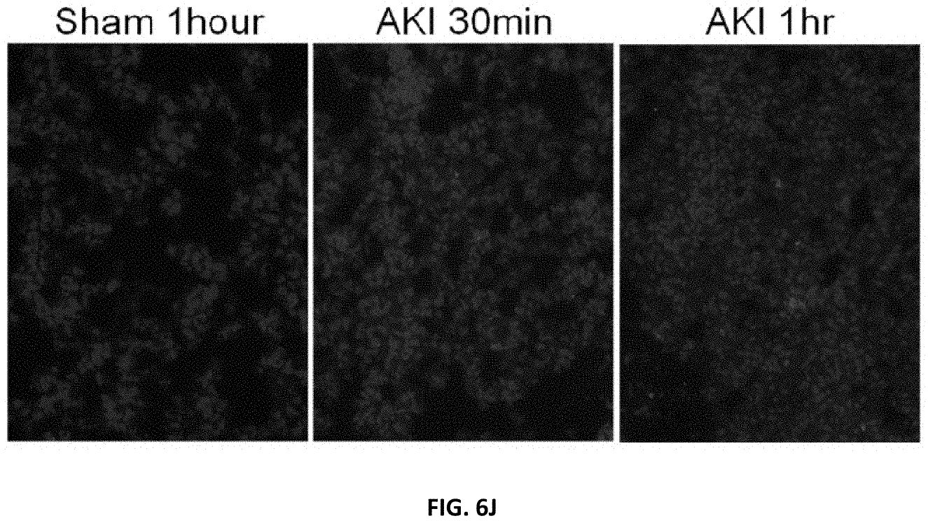

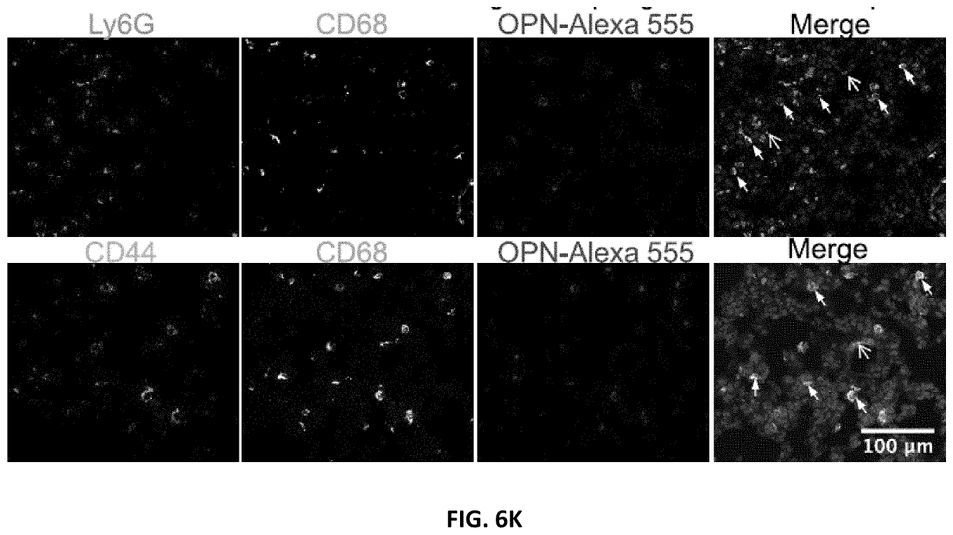

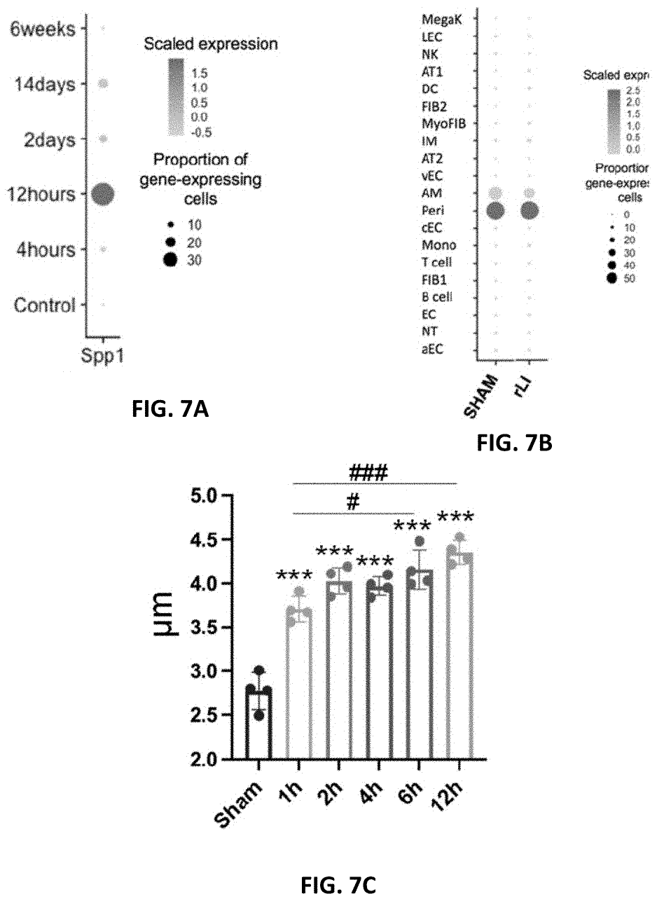

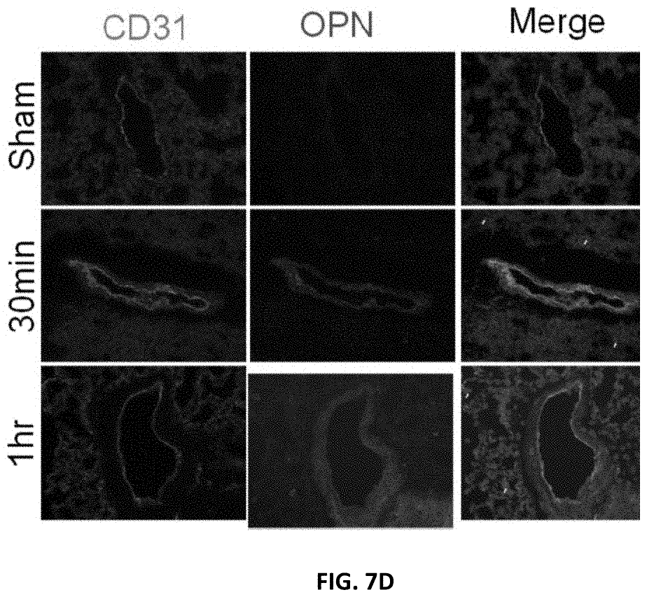

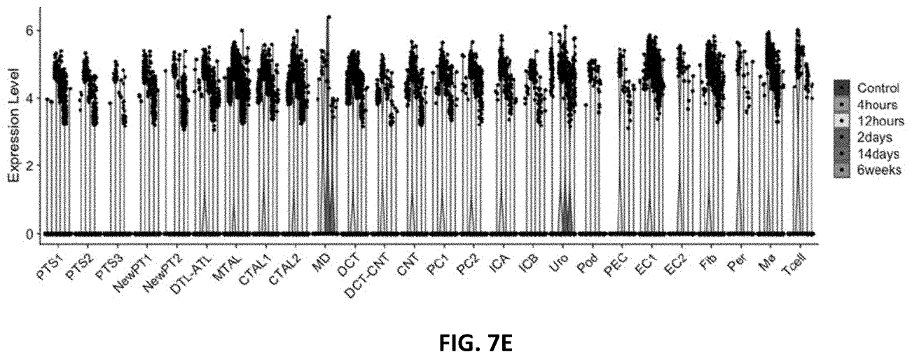

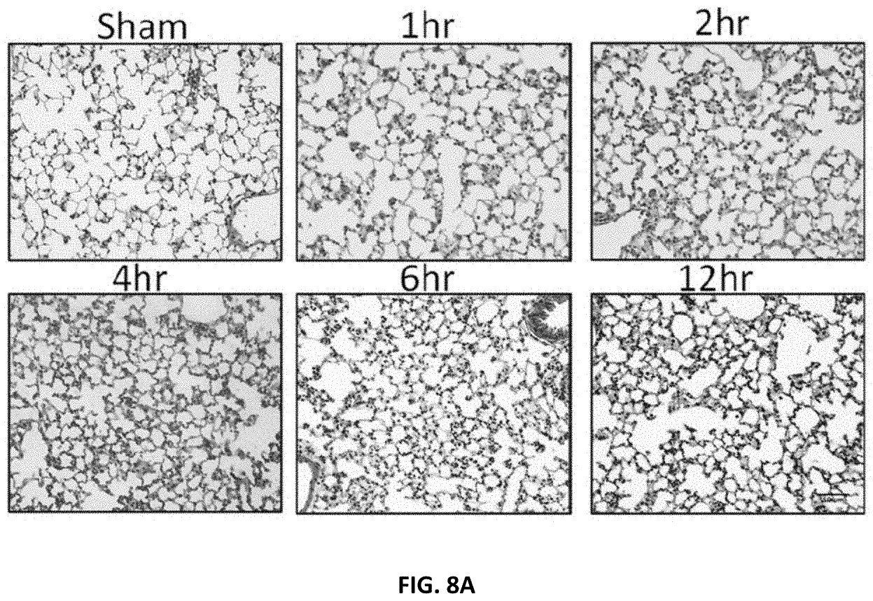

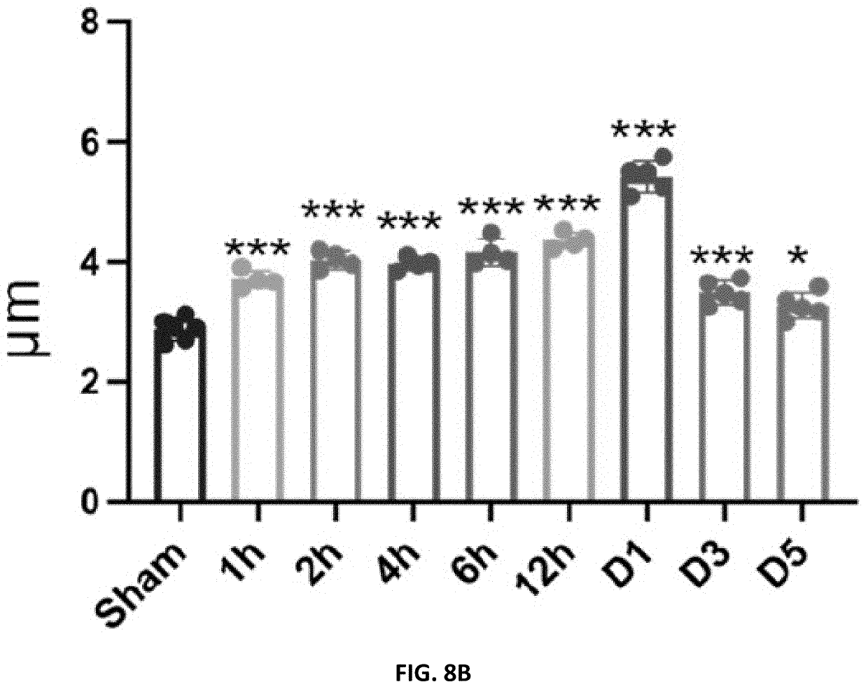

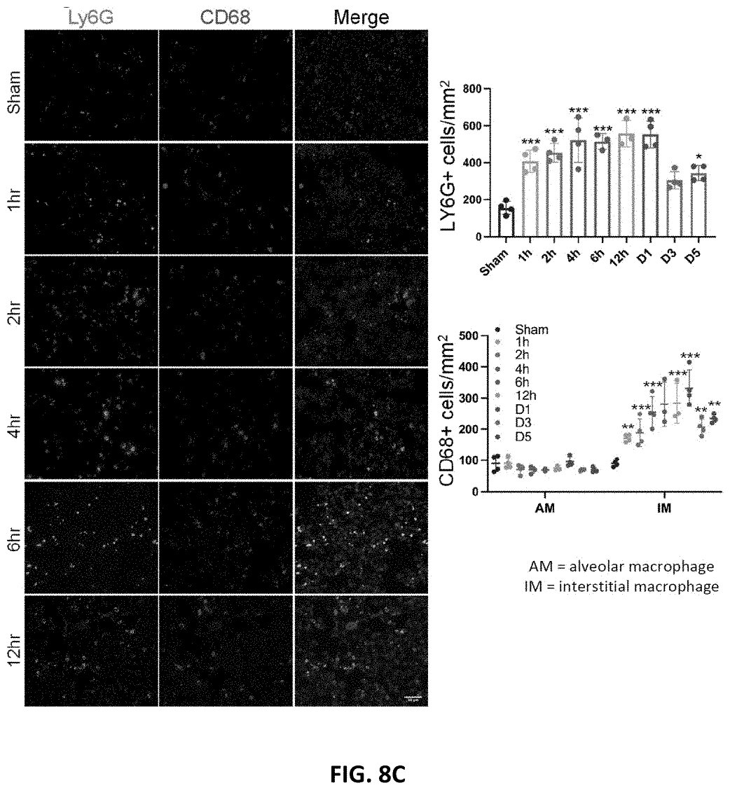

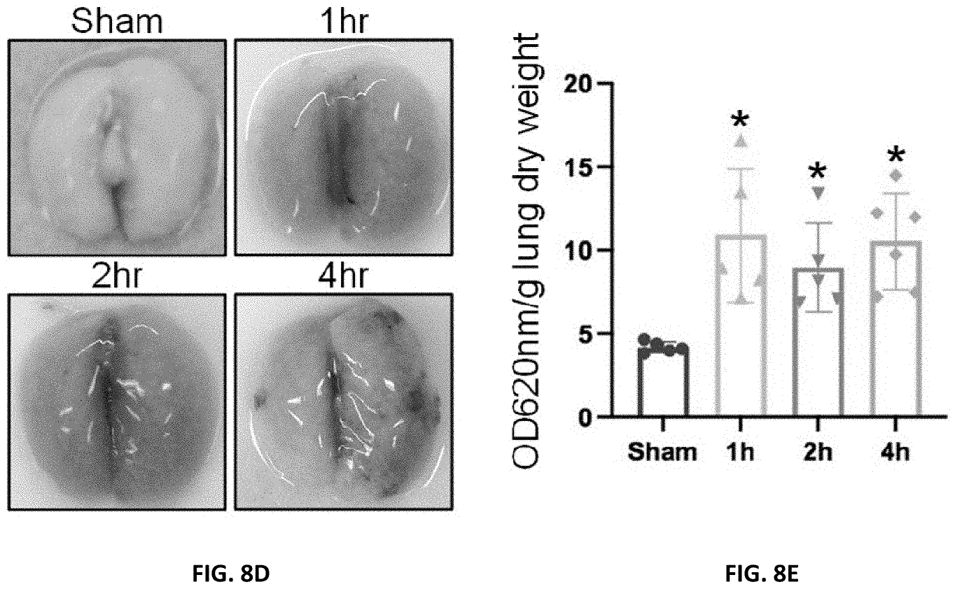

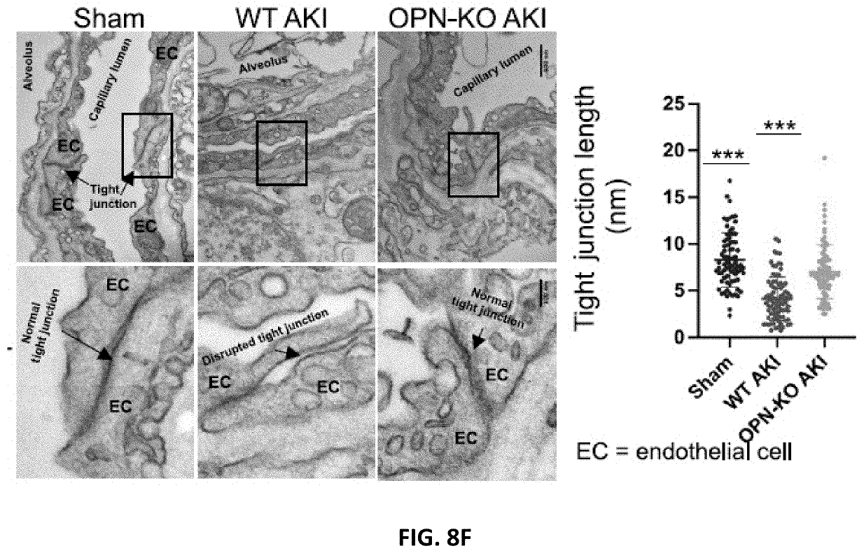

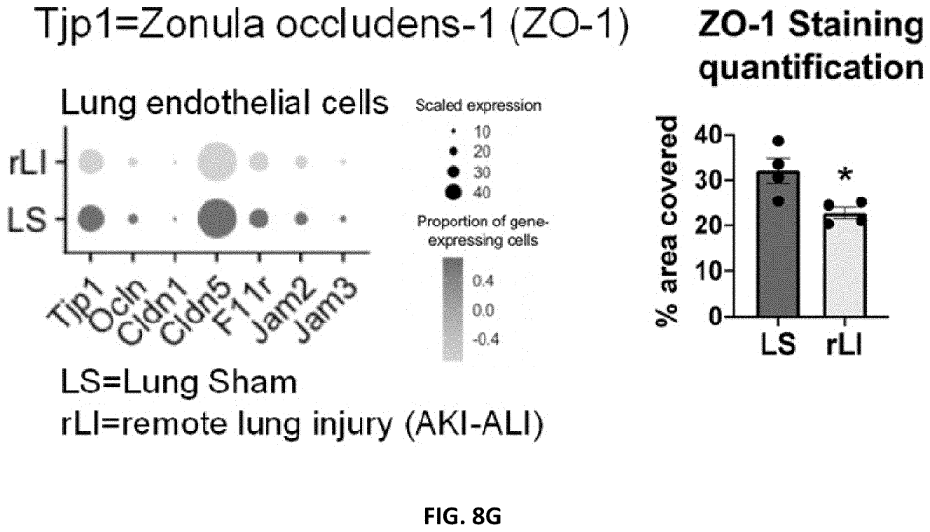

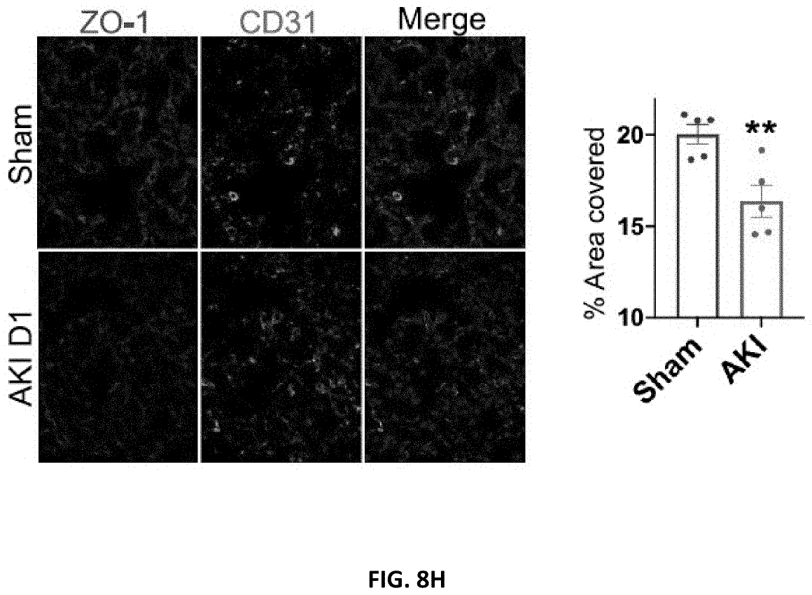

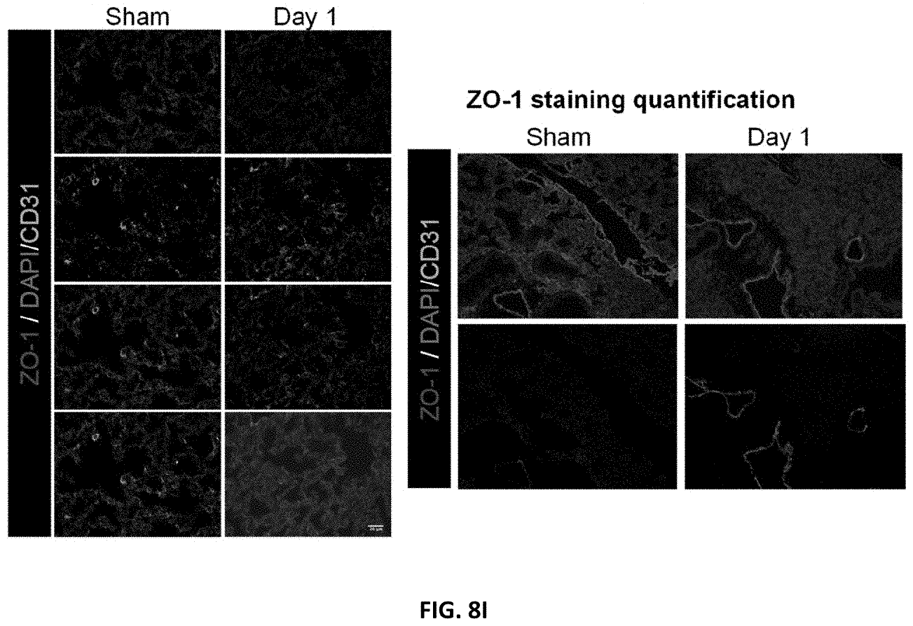

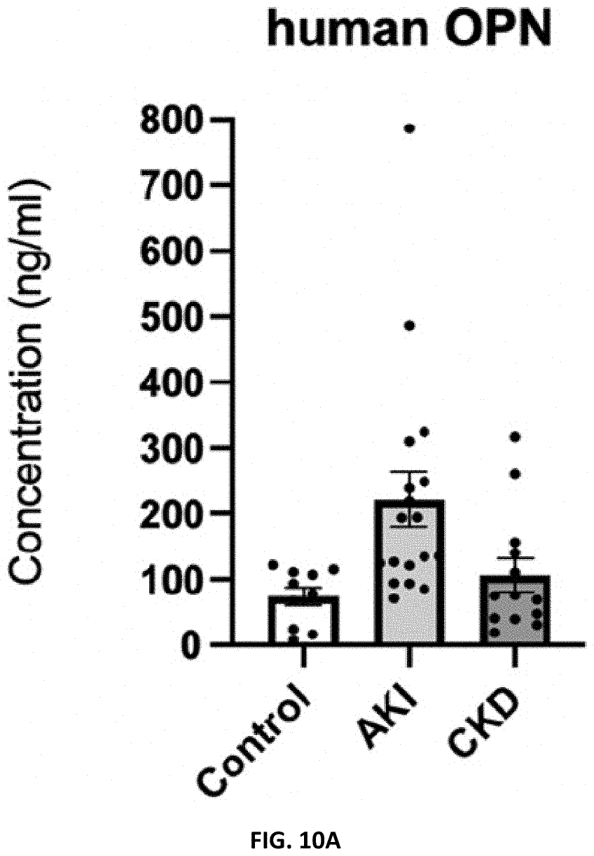

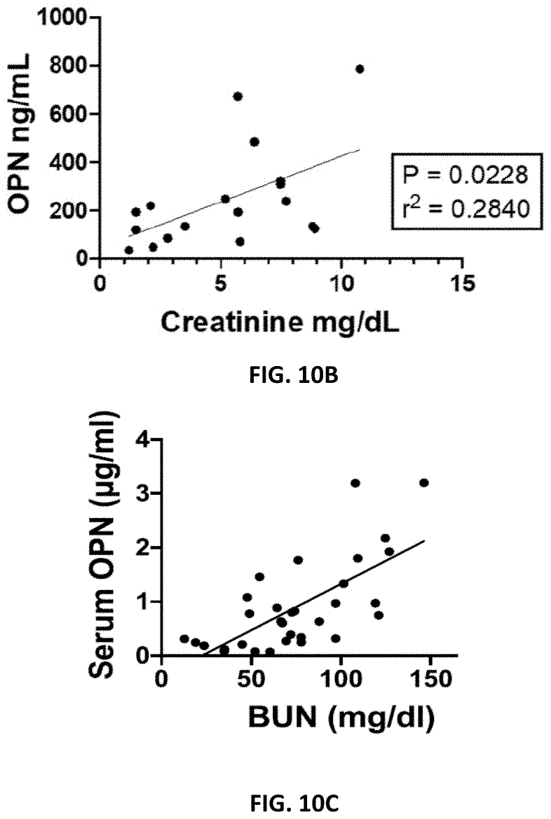

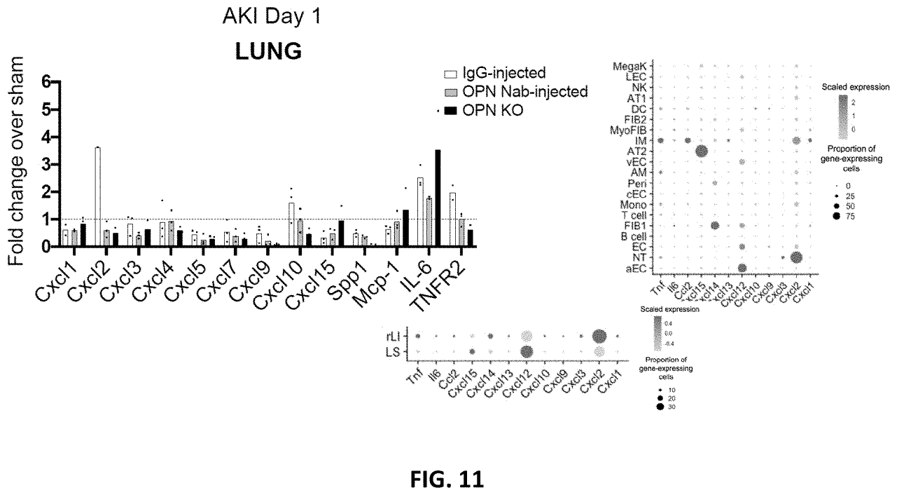

To understand the relationship between OPN and the development of AKI-ALI, expression patterns of OPN mRNA and OPN protein serum levels during the course of AKI was assessed. On the basis of our scRNAseq data, OPN mRNA is already expressed at significant levels in distal tubule and to a lesser degree in proximal tubule cells of sham kidneys (FIG. 6A, top). On day 1 after AKI, OPN expression was not only most significantly increased in both proximal and distal tubule cells but, now, also present at lower levels in other cell types such as endothelial cells, fibroblasts, neutrophils, macrophages, and T cells (FIG. 6A, bottom). This suggests that tubule-released OPN could potentially act in the induction phase of AKI-ALI and that, at later stages, various cellular OPN sources, including immune cells, might contribute to elevated OPN serum levels. These findings were validated by comparing the scRNAseq data with previously published snRNAseq AKI data. Similar to the dataset of this study, OPN mRNA expression in sham kidneys was present in both proximal tubule cells (PTC and new PTC) and distal tubule cells [CNT (connecting tubule), descending-type thin limb-ascending-type thin limb (DTL-ATL), DCT (Distal Convoluted Tubule), and Uro (Urothelium)]. By 4 hours after AKI, OPN mRNA expression began to increase significantly in several cell types, and by 12 hours, OPN mRNA expression was very high in proximal and distal tubules, endothelial cells, fibroblasts, neutrophils, macrophages, and T cells, similar to the dataset on day 1 after AKI. OPN expression remained elevated for several weeks in distal tubular compartments (FIG. 6B). In contrast to the kidney, OPN expression in sham lung was only present in moderate levels in pericytes and, to a lesser degree, in resident alveolar macrophages. AKI injury did not change the overall low OPN expression levels detected by scRNAseq in the lung on day 1 after AKI (FIG. 6C, top and bottom), suggesting that the kidney but not the lung represents a substantial source of OPN after AKI. To better understand dynamic changes in our model, additional AKI-ALI experiments, evaluating earlier time points, were performed. C57BL/6 mice were subjected to AKI and euthanized them 1, 2, 4, 6, and 12 hours later (FIG. 6D, experimental scheme). In FIGS. 6 and 8, AKI-ALI data from day 1, 3, or 5 after AKI derived from FIG. 1 are incorporated for ease of viewing. Serum BUN and serum creatinine levels were already significantly elevated 1 hour after AKI (FIGS. 6E and 6F), and serum kidney injury molecule-1 (KIM-1; a sensitive marker of kidney injury) was elevated at 12 hours (FIG. 6G), indicating substantial kidney injury. Kidney OPN expression was already significantly elevated 2 to 4 hours after AKI, reached a peak by 12 hours, and remained elevated until at least day 5. Lung OPN expression, however, remained virtually undetectable or very low over the same time (FIG. 6H). Serum OPN protein levels closely followed changes in kidney OPN mRNA expression (FIG. 6I). The early rise in OPN serum levels is likely enabled by the existing baseline expression of OPN in the sham kidney (FIGS. 6A and 6B). Elevated OPN levels in AKI-injured mice correlated with the degree of kidney injury (BUN/OPN) (FIG. 10C).

Serum OPN protein levels have been studied as a biomarker of severity of disease in patients with multiorgan failure, often including AKI and ALI. Whether serum OPN levels are also elevated in patients with AKI before development of multiorgan failure is not known. OPN serum levels in patients with AKI that did not have multiorgan failure and were not critically ill were examined. Serum OPN levels were significantly elevated in patients with AKI, as compared to healthy controls or patients with chronic kidney disease (CKD) (FIG. 10A) and positively correlated with reduced kidney function as determined by serum creatinine measurements (FIG. 10B). These findings suggest that AKI in humans could result in release of substantial amounts of OPN into the circulation and raise the possibility that circulating OPN may promote damage at remote sites also in humans.

It was hypothesized that for OPN to act on lung and cause AKI-ALI, it might need to gain access to the lung tissue compartment from the circulation. Alternatively, it could act on cells directly accessible from the circulation, such as endothelial cells or circulating immune cells. Whether OPN can gain access to lung tissue from the circulation in the setting of AKI in mice was thus examined. Fluorescently labeled OPN was injected after sham operation or after AKI at the time of reperfusion and euthanized animals 30 min or 1 hour after injection. The samples were costained for nidogen, a marker of the extracellular matrix/interstitial space. Sham-operated animals did not accumulate any significant amount of fluorescently marked OPN in the lung within 1 hour of injection, whereas animals with AKI showed OPN accumulation in lung interstitium (thin arrows) and colocalization with lung cells (thick arrows) at 30 min or 1 hour after injection (FIG. 19). Ly6G, CD68, and CD44 costains reveal that injected fluorescently marked OPN colocalizes with CD68+ alveolar macrophages and IMs (which also show the highest staining for CD44) but not with Ly6G-positive neutrophils (FIG. 6K). Together, the results indicate that kidney rather than lung represents the source of circulating OPN protein after AKI and that circulating OPN can principally gain access to the lung early after AKI and thus might mediate the induction of remote lung injury. As predicted from the L-R pairing analysis in FIG. 3, CD44 in lung immune cells, particularly alveolar macrophages and IMs, may represent important targets of OPN released from the kidney. Access to the lung was marginal in sham-operated animals, leading to the conclusion that another AKI-released factor must cooperate with OPN to allow OPN access to lung tissue.

AKI Triggers Lung Endothelial Barrier Dysfunction and Immune Cell Accumulation Very Early after Injury

Consistent with the timing of elevated OPN serum levels after AKI, lungs already showed significant signs of injury with interstitial expansion and increased cellularity by 1 hour after AKI, which worsened progressively over the subsequent 12 hours (FIG. 8A, also compare to FIG. 1D). Alveolar wall thickness increased significantly by 1 hour (>1.3-fold increase over sham), worsened progressively over the ensuing 12 hours (>1.5-fold increase over sham), and reached a peak at day 1 after AKI (>1.85-fold increase over sham) (FIG. 8B). Consistent with these findings, accumulation of neutrophils and macrophages showed a similar trend to the timeline of changes in kidney OPN mRNA expression and serum OPN levels (FIG. 8C).

Endothelial homeostasis is disrupted in ALI or its most severe form, ARDS, which often occurs in response to direct lung injury, such as by coronavirus disease 2019 (COVID-19) lung infection, or as a secondary organ complication of sepsis. Severe ALI/ARDS is characterized by diffuse endothelial injury, intense activation of the coagulation system, and increased capillary permeability. Increased vascular barrier permeability has also been reported in animal models of AKI. Whether endothelial barrier disruption, a hallmark of lung injury, also occurs following AKI in our model was examined. Vascular leakage, as evidenced by Evans blue dye uptake, was already detectable at a level twice as high as sham controls at 1 hour after AKI, which was maintained for at least 4 hours (FIGS. 8D and 8E). Electron microscopy also showed that, compared to sham controls, endothelial tight junctions in AKI-injured C57BL/6 animals were shortened in length or disrupted in day 1 AKI-ALI lungs. By contrast, AKI-injured OPN-global KO mice displayed normal endothelial tight junctions, comparable to sham-operated wt animals (FIG. 8F). Lung endothelial cells were assessed next. Using our scRNAseq data, it was found that the expression levels of two endothelial barrier proteins, zonula occludens-1 [ZO-1; Tjp1 (tight junction protein 1)] and claudin-5 (Cldn5), were strongly down-regulated in AKI-ALI samples as compared to sham lungs (FIG. 8G). The finding on the protein level for ZO-1 was validated. ZO-1 staining was heavily present in the capillary network of sham lung but severely diminished in the capillary network of the AKI-ALI lung. CD31 (green) was used as a costain to identify endothelial cells (FIG. 8H). Other cell types in the lung, particularly stromal cells, such as fibroblasts and lung epithelial cells (AT1/2), also expressed ZO-1 or Cldn5 at low levels, and these expression levels did not significantly change in the AKI-ALI lung. These results suggest that endothelial barrier dysfunction and vascular leakage represent early events in the induction of AKI-ALI remote lung injury in our model.

Pharmacological or Genetic Inhibition of OPN Protects from ALI after AKI

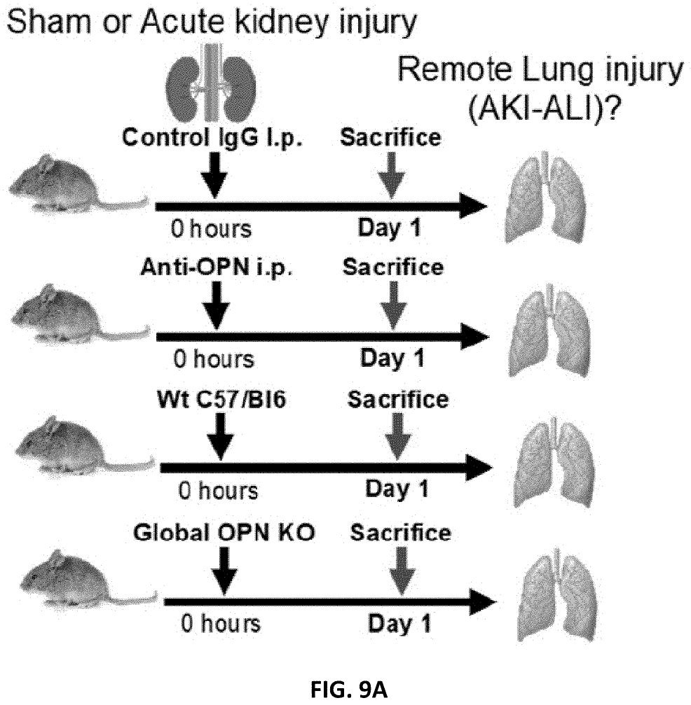

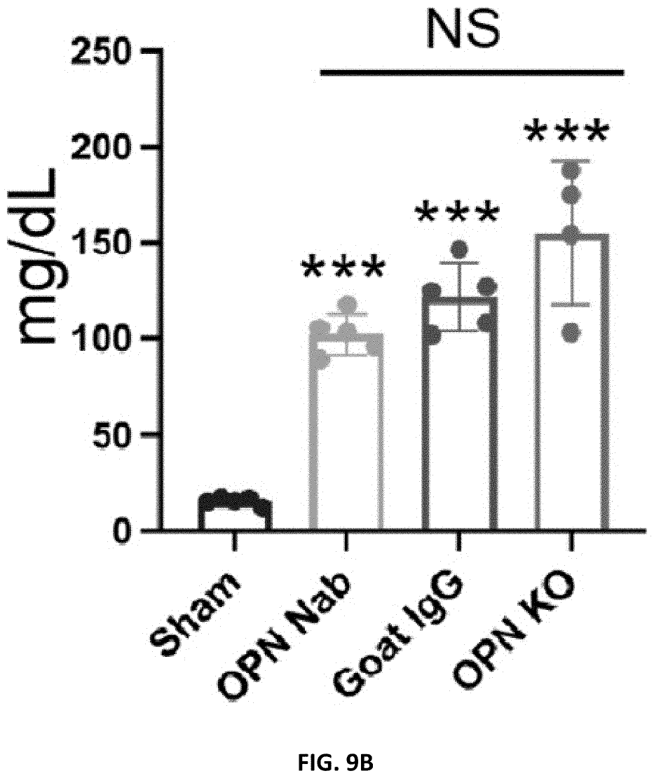

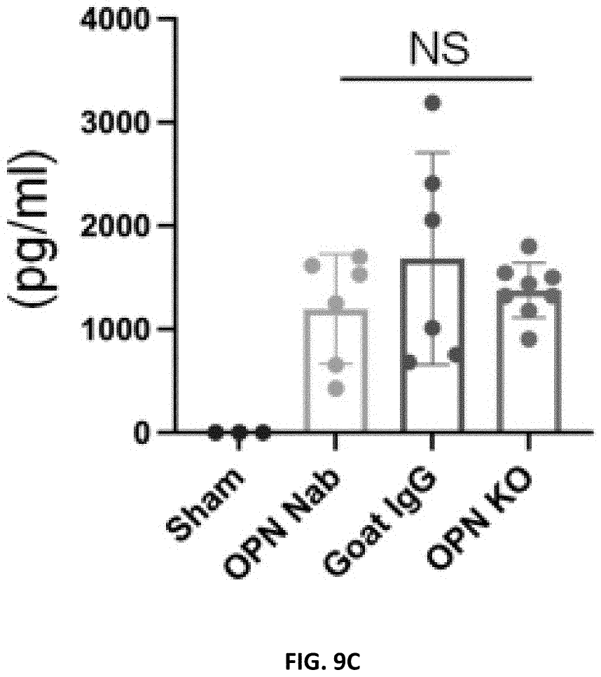

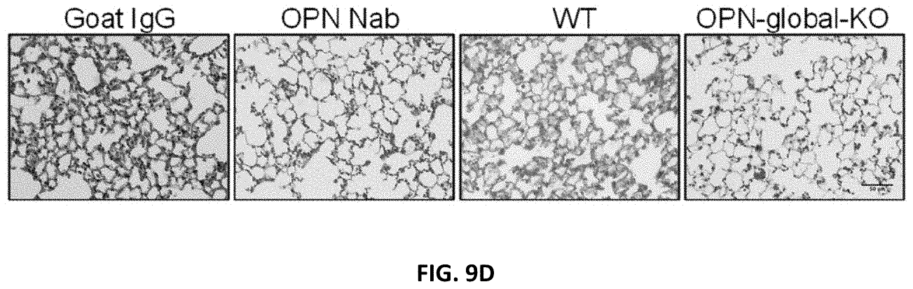

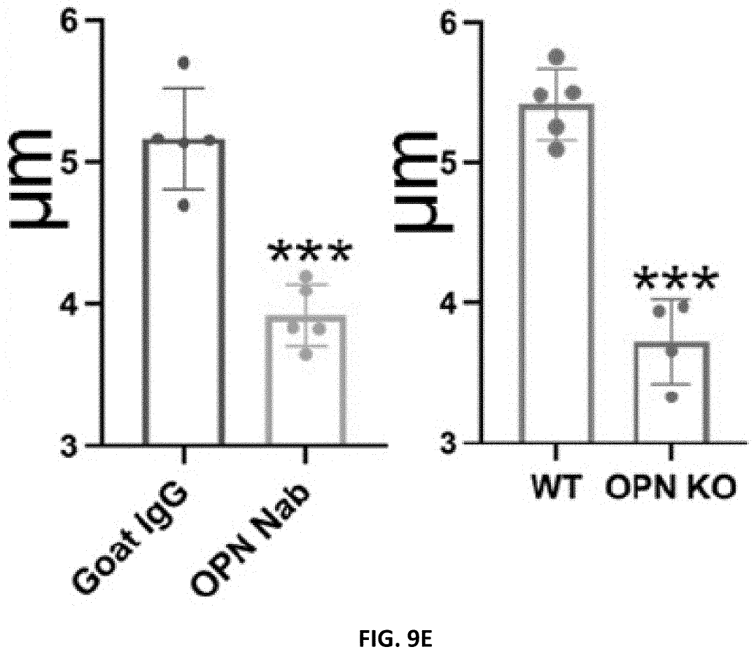

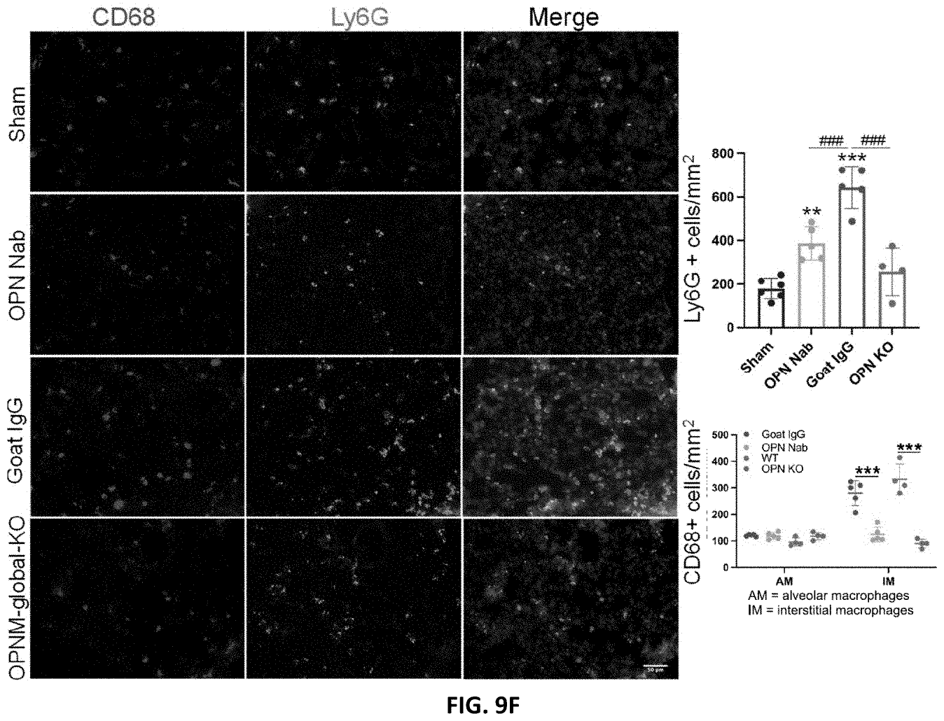

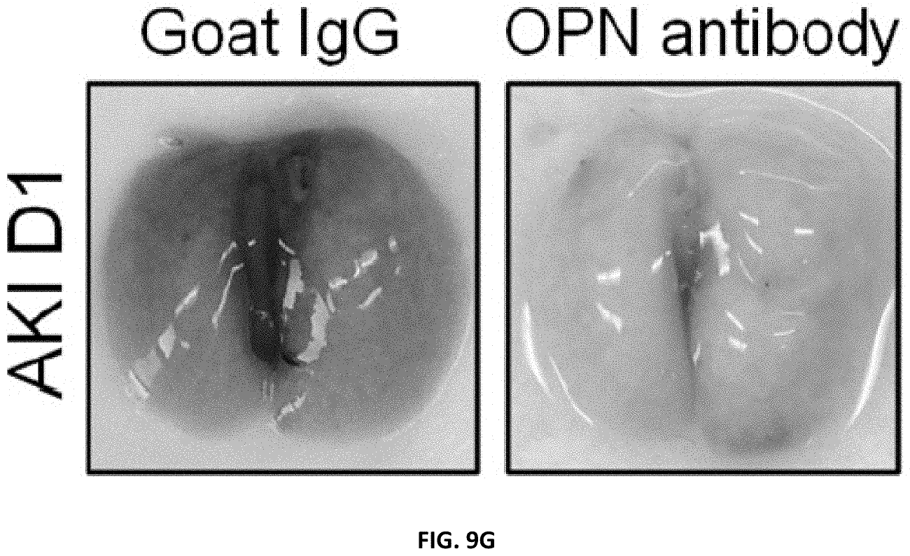

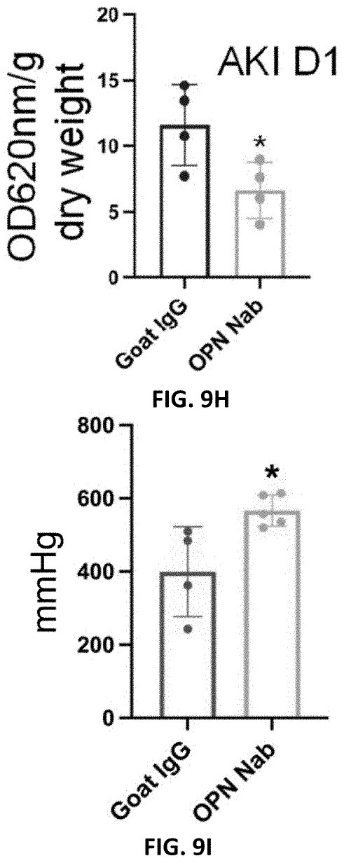

To assess whether OPN is necessary for the development of AKI-ALI, C57BL/6 mice were subjected to bilateral IRI and injected the animals with either control immunoglobulin G (IgG) or anti-OPN neutralizing antibody. Analogous experiments were performed in OPN-global KO or wt mice without antibody treatments (FIG. 9A, experimental scheme). Anti-OPN antibody-treated mice or OPN-global KO mice experienced the same degree of kidney injury on day 1 as controls, as evidenced by comparable elevations in serum BUN and KIM-1 levels (FIGS. 9B and 9C). However, lung injury was significantly ameliorated after treatment with anti-OPN antibody and in OPN-global KO mice (FIGS. 9D and 9E). While anti-OPN antibody strongly reduced lung neutrophil and IM accumulation after AKI, OPN-global KO prevented it almost completely (FIG. 9F). Anti-OPN antibody-treated animals also showed strongly reduced vascular leakage and improved lung function as compared to controls on day 1 after AKI (FIG. 9G, 9H, 9I). Together, the results identify circulating OPN as a critical and necessary regulator of lung endothelial barrier permeability, lung immune cell accumulation, and functional impairment in AKI-ALI.

Circulating OPN is Sufficient to Induce ALI after AKI

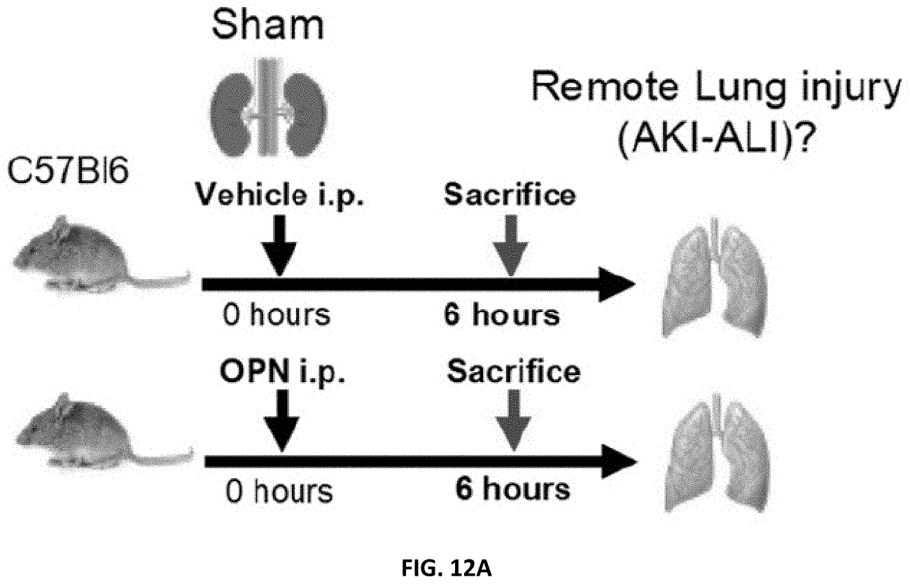

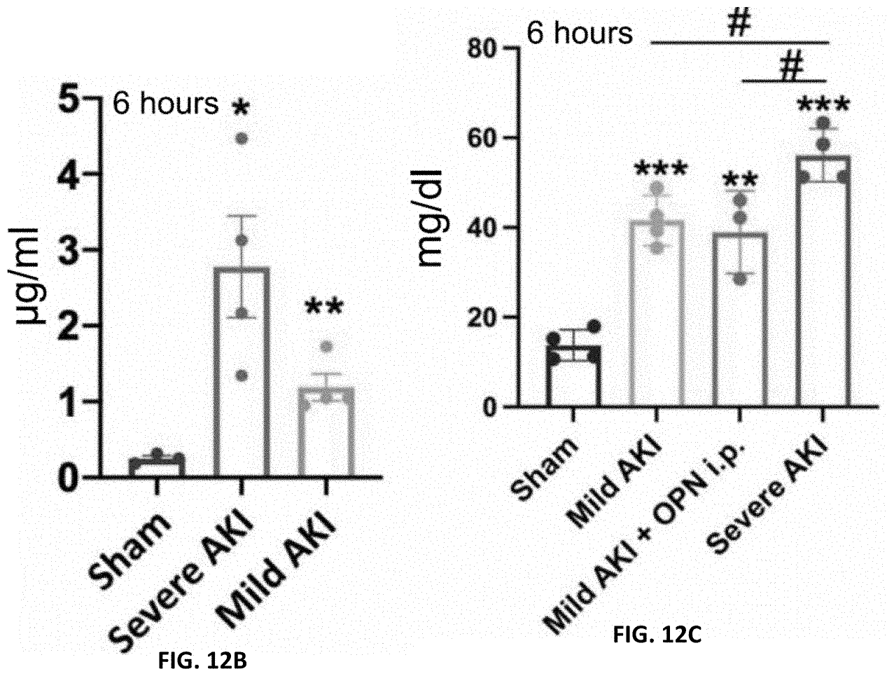

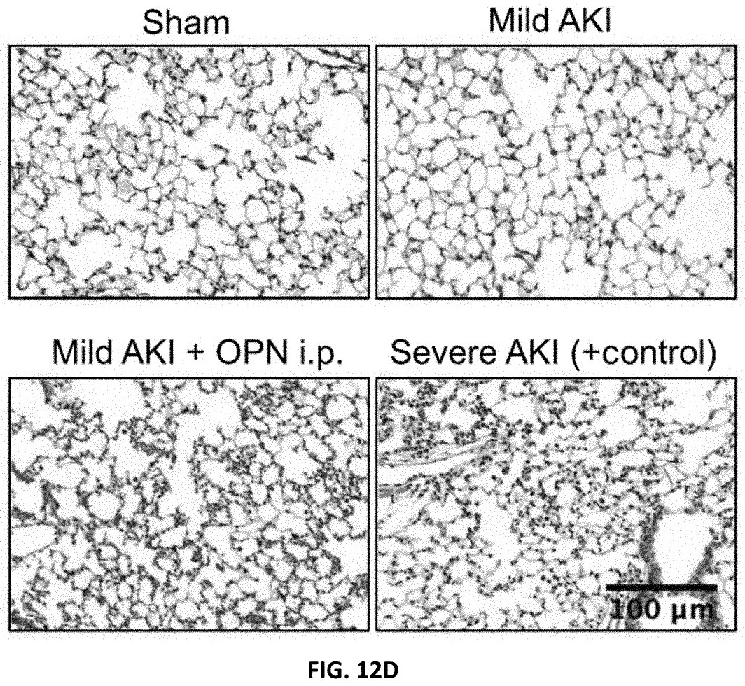

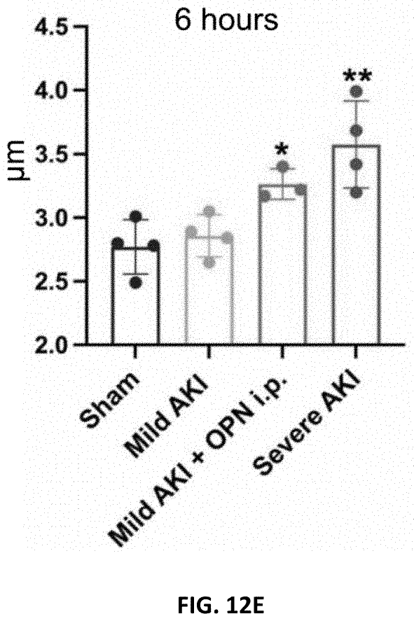

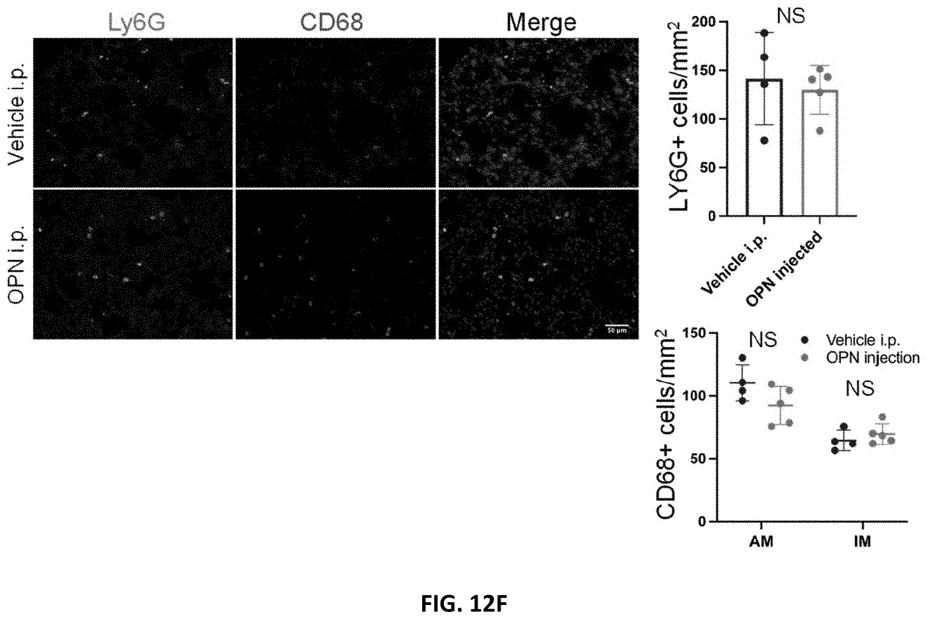

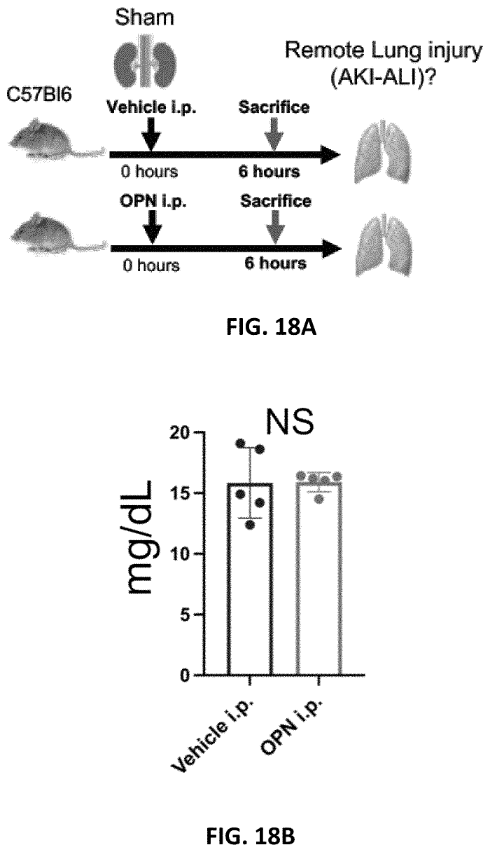

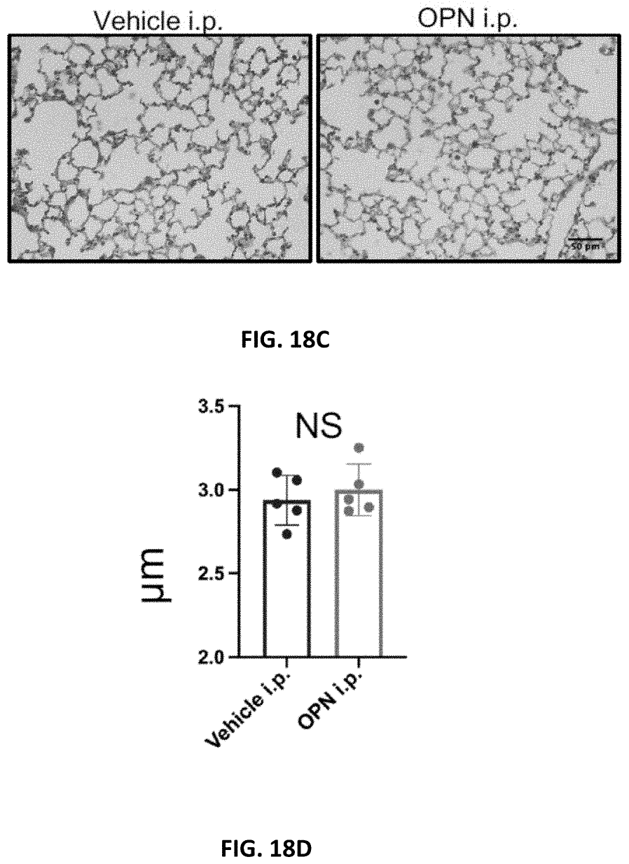

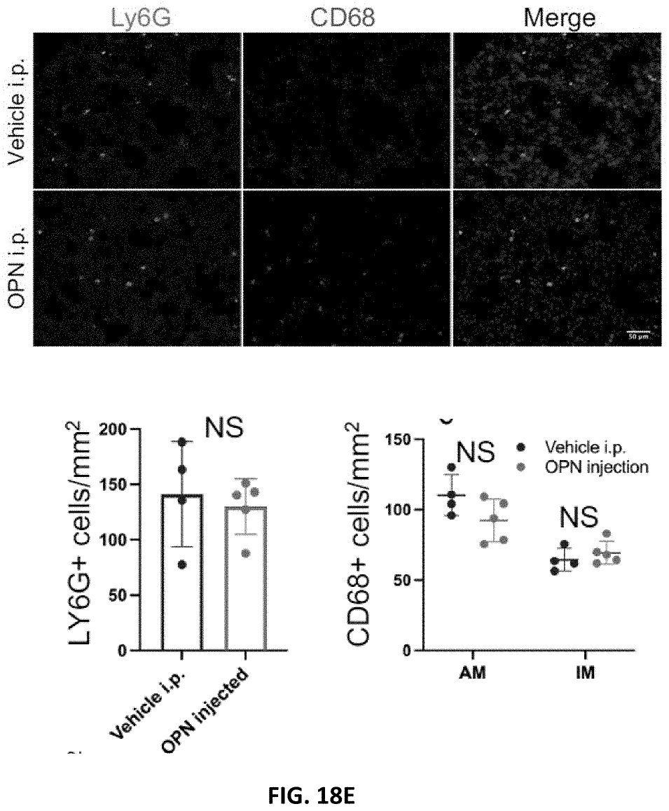

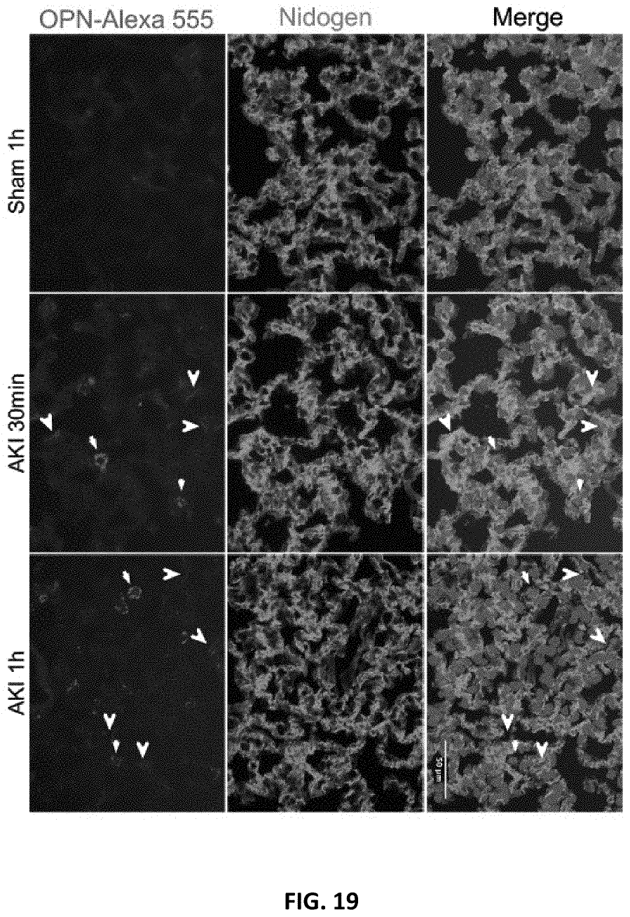

To determine whether OPN suffices to mediate AKI-ALI, the effect of OPN injection was tested in the context of mild AKI (reduced ischemia time), an experimental condition where AKI-ALI is not detectable at 6 hours after AKI (FIG. 12A, experimental scheme). As expected, serum OPN levels were significantly lower in mild AKI than severe AKI (FIG. 12B). Serum BUN levels were significantly elevated at 6 hours after mild AKI compared to sham controls but lower compared to severe AKI (FIG. 12C). Intravenous injection of OPN protein into mild AKI animals at the time of kidney reperfusion at quantities that would mimic serum concentrations at 6 hours after severe AKI (see FIG. 6I) triggered AKI-ALI that was now comparable to severe AKI-ALI (FIGS. 12E and 12F). OPN injections into uninjured mice were also performed but increases in alveolar wall thickness or inflammatory changes in the lung were unable to be detected (FIGS. 18A, 18B, and 18C), suggesting that a second event or mediator induced by AKI is required for OPN to successfully induce AKI-ALI. Therefore, it is concluded that OPN is sufficient to induce AKI-ALI only in the context of AKI.

Circulating OPN is Relevant for Induction of AKI-ALI is Released from the Injured Kidney

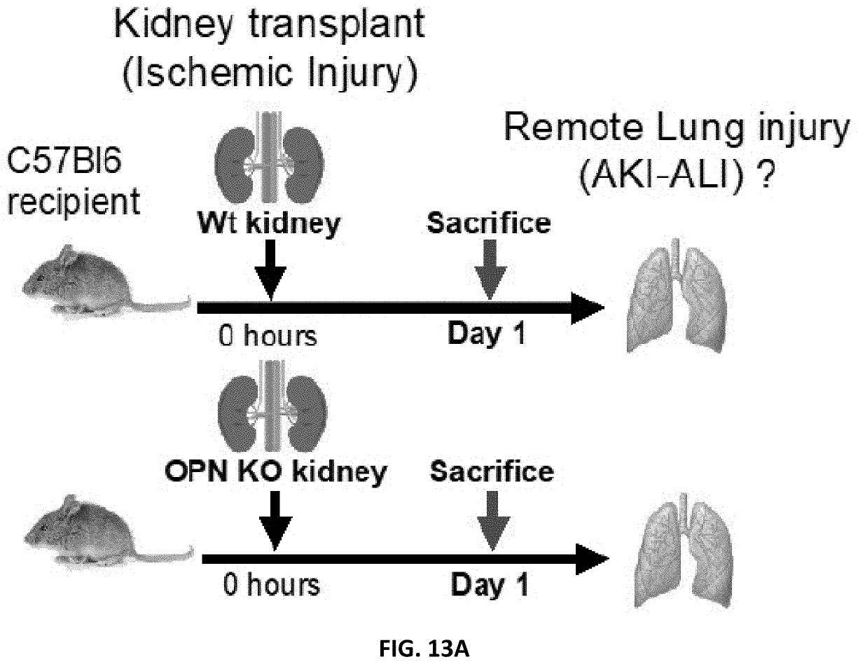

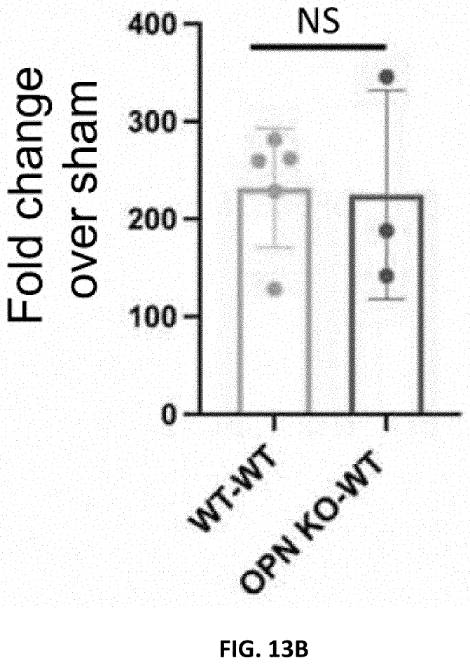

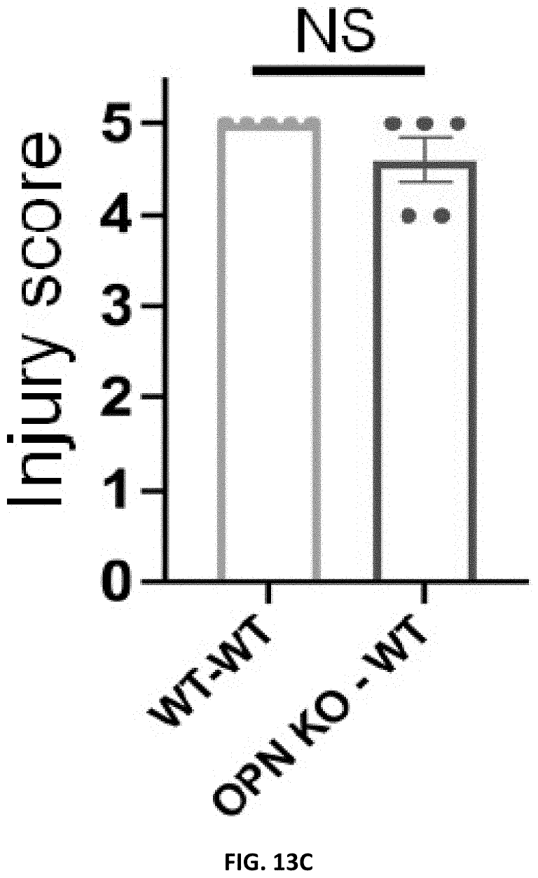

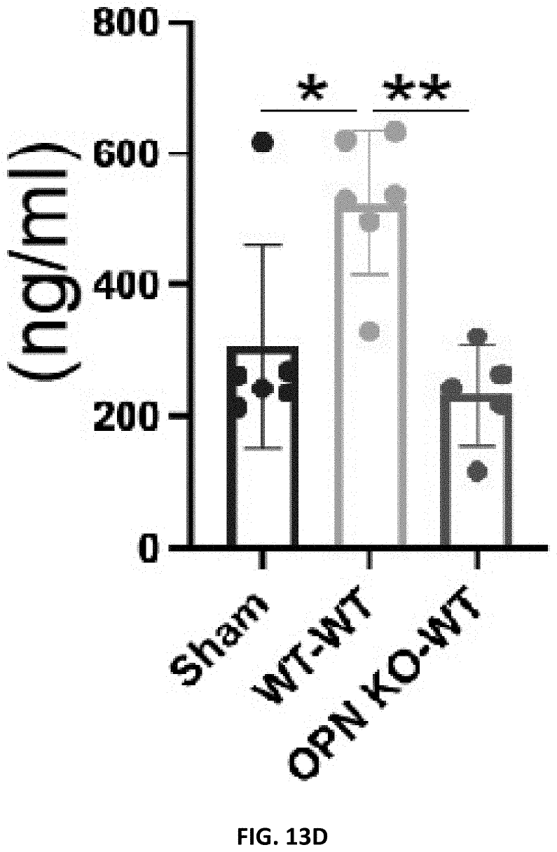

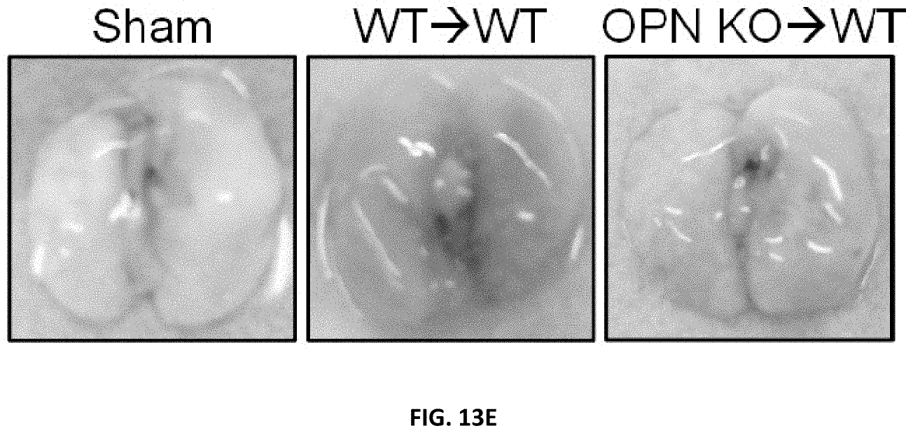

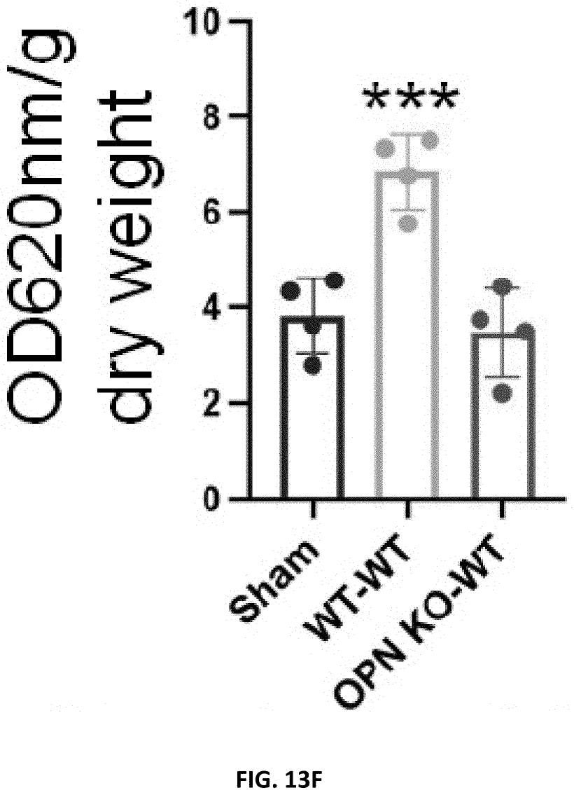

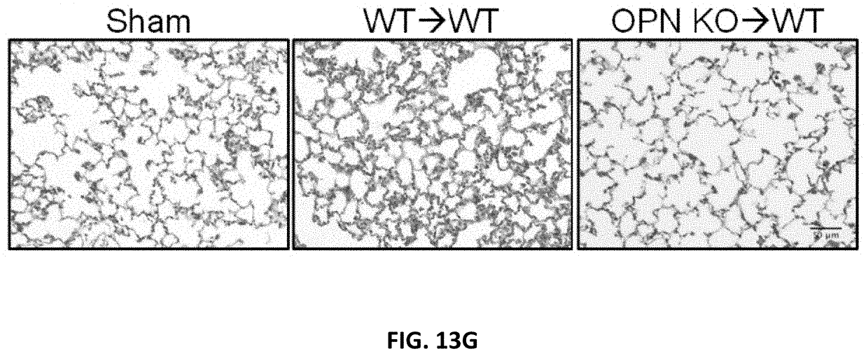

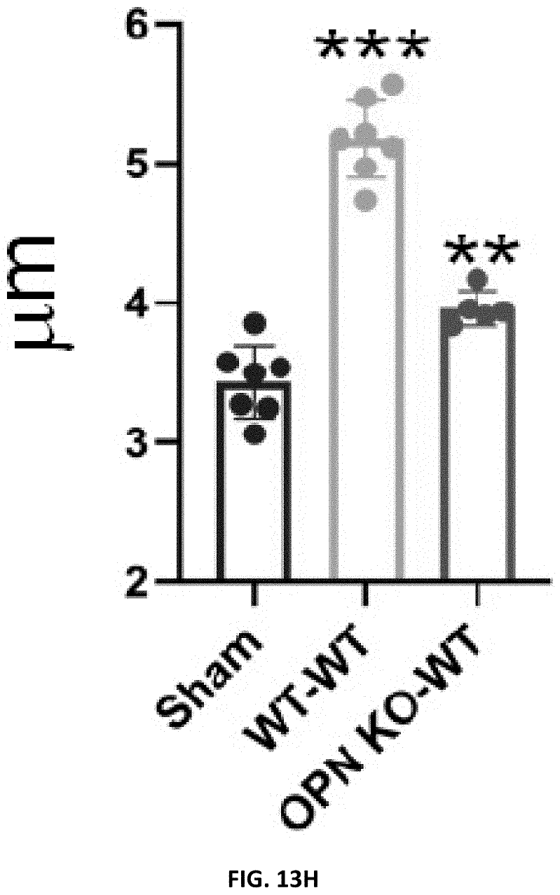

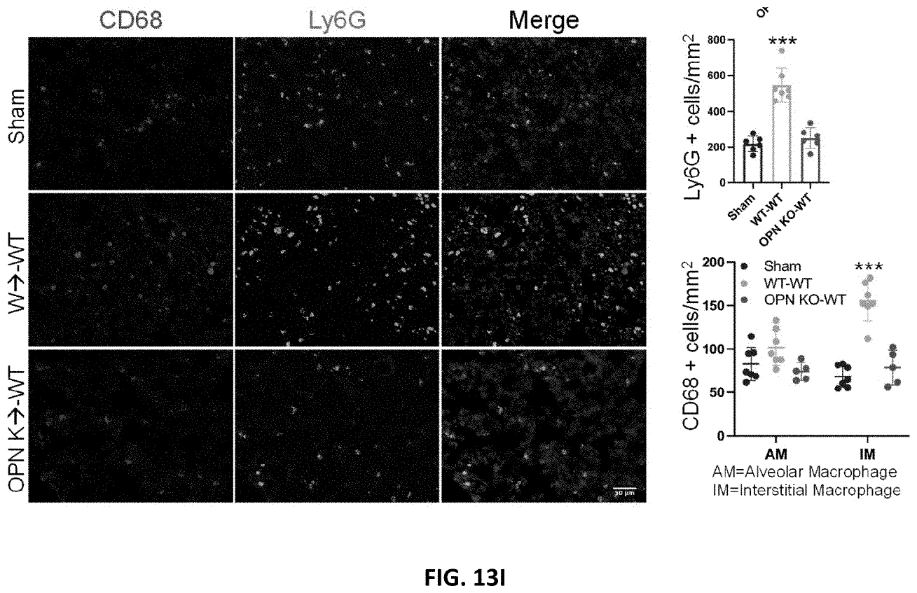

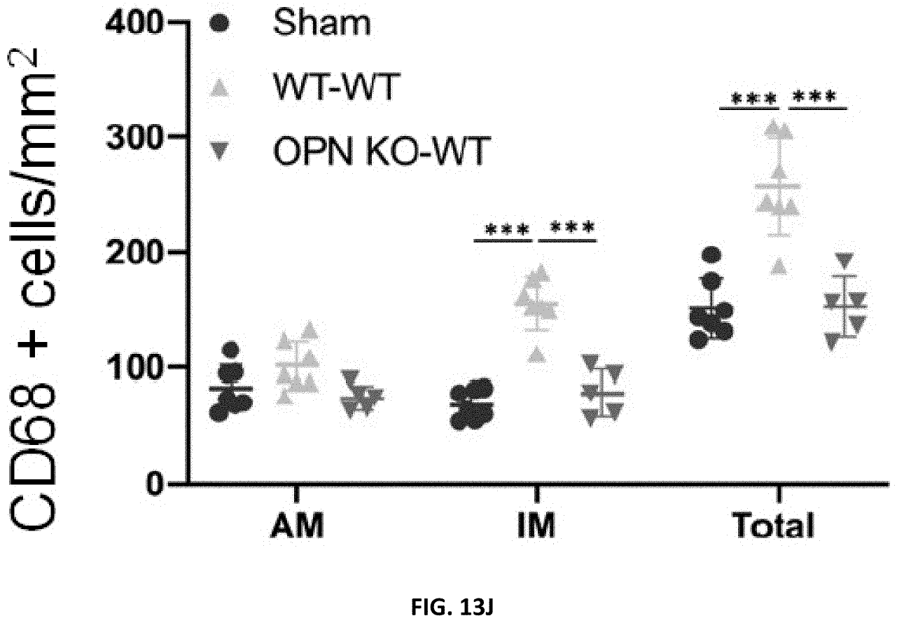

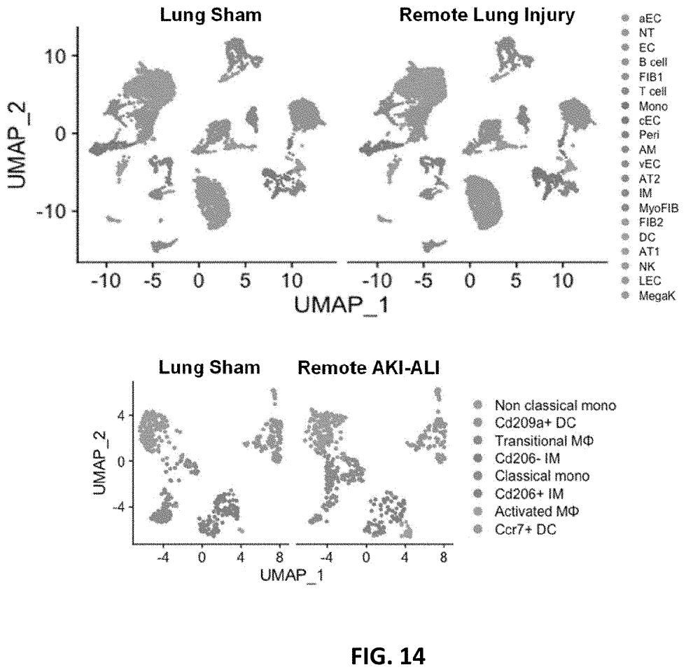

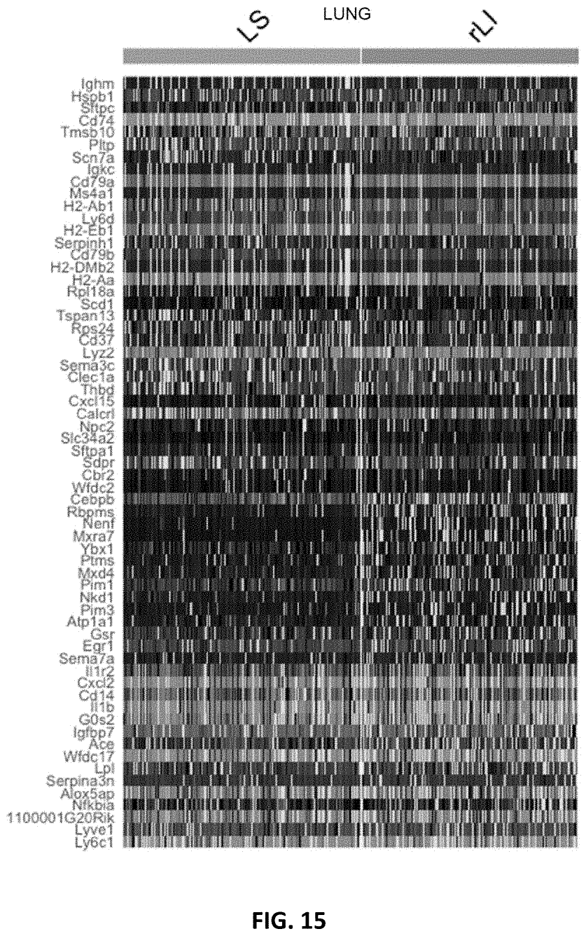

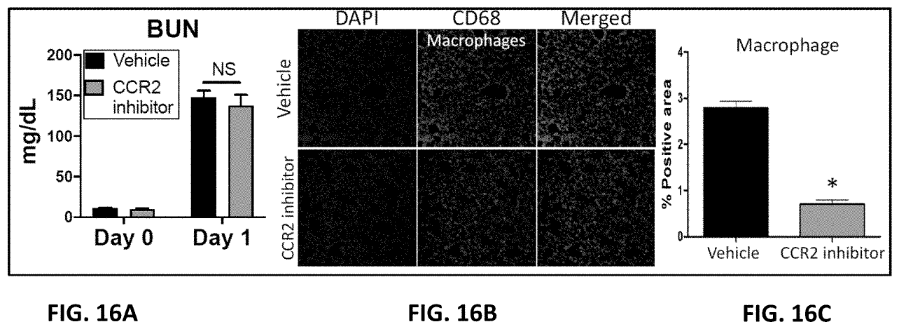

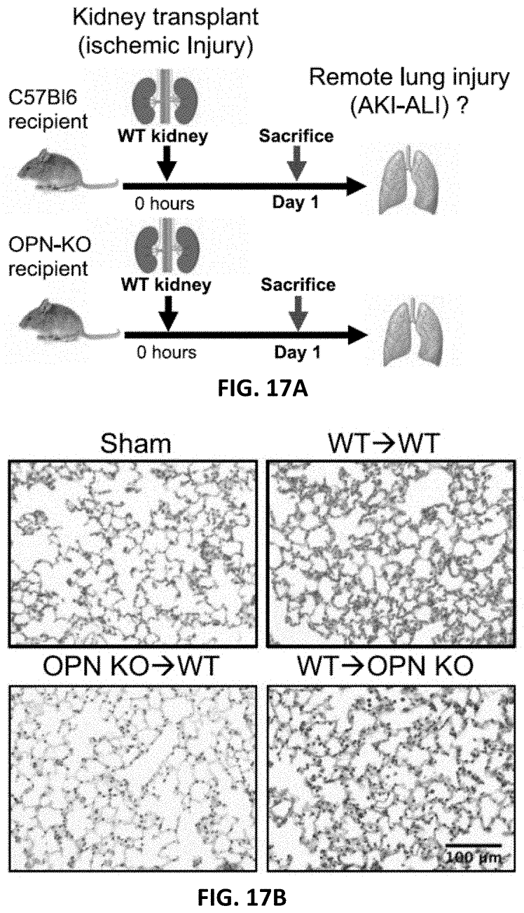

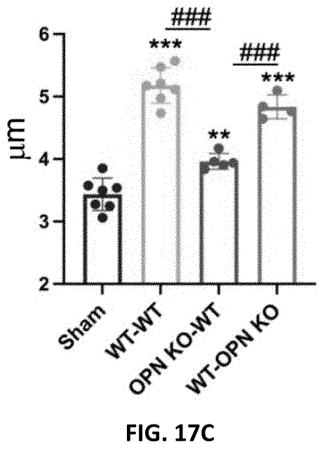

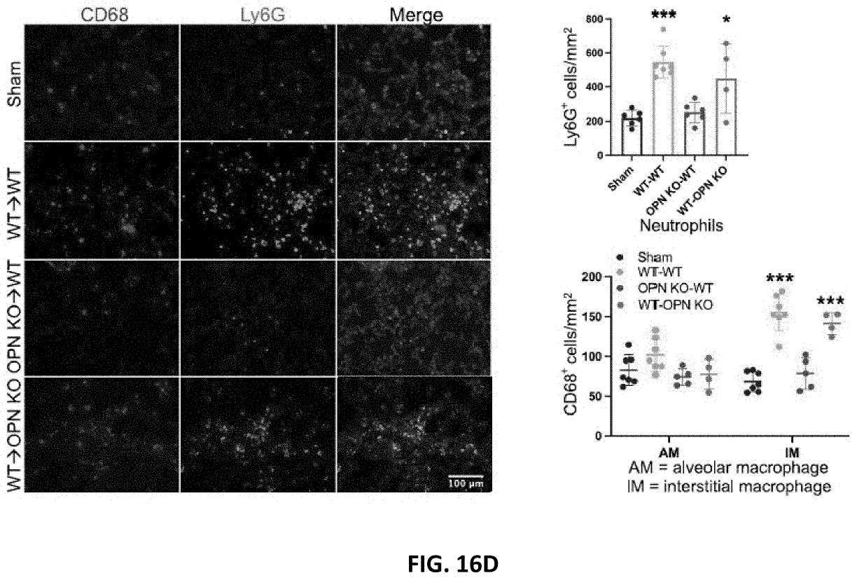

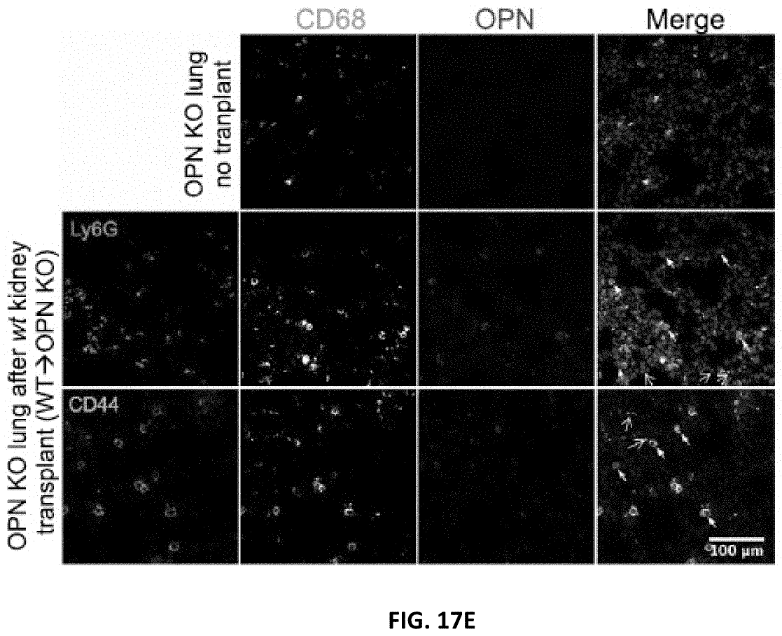

Up-regulated OPN expression and serum levels have been found in the context of a number of organ injuries, allowing for the possibility that OPN sources in the body other than the kidney might be relevant in AKI-ALI. Conversely, it has never been conclusively shown that a proposed AKI-ALI mediator is released directly from the injured kidney. To address this question, kidney transplantation experiments were performed, where either ischemic wt kidneys or ischemic OPN-global KO kidneys were transplanted into syngeneic C57BL/6 mice (FIG. 13A, experimental scheme). KIM-1 kidney mRNA expression and tubular injury scores document that transplanted OPN-global KO kidneys show kidney injury parameters comparable to those in transplanted wt kidneys (FIGS. 13B and 13C). Serum BUN and creatinine measurements would be unaffected in the transplantation model, given that two intact kidneys are present in the mouse as well. OPN serum levels were elevated above sham levels in mice that received wt kidneys but not in mice that were transplanted OPN-KO renal grafts. Notably, OPN serum levels are lower with only one injured transplant kidney, as compared to the bilateral IRI model, where two kidneys are injured (FIG. 13D; see also FIG. 6). Lung injury developed in mice that received wt kidneys but not in those that received OPN-KO kidney grafts (FIGS. 13E, 13F, 13G, 13H, and 13I). These results conclusively identify the kidneys as the source of OPN during the development of ALI after AKI. Last, when ischemic wt kidneys are transplanted into OPN-global KO mice (FIG. 17A), the protection of the latter from developing AKI-ALI is reversed, and these mice develop AKI-ALI similar to wt mice transplanted with ischemic wt kidneys (FIG. 17B). Alveolar wall thickness increases (FIG. 17C), and infiltration of immune cells is now equal to the effect of wt kidney transplant into wt mice (FIG. 17D). OPN released from the transplanted wt kidney is detectable in the lung and colocalizes with CD68+ and CD44+ alveolar macrophages and IMs but not with Ly6G+ neutrophils (FIG. 17E), identical to exogenously injected OPN (see FIG. 6K). These results confirm the critical necessity for OPN in the development of AKI-ALI and suggest again that OPN may act on CD44-expressing macrophages in the lung.

What is claimed is:

1. A method of treating or preventing an acute kidney injury-induced acute lung injury (AKI-ALI) in a subject in need by administration of a therapeutically effective amount of an osteopontin inhibitor.

2. The method of claim 1, wherein the osteopontin inhibitor comprises an anti-OPN-antibody.

3. The method of claim 1, wherein the administration of the therapeutically effective amount of the osteopontin inhibitor results in inhibition of lung vascular leakage and inflammation.

4. A method of treating or preventing a multi-organ failure associated with an acute tissue injury in a subject in need by administration of a therapeutically effective amount of an osteopontin inhibitor.

5. The method of claim 4, wherein the osteopontin inhibitor comprises anti-OPN-antibody.
